(19) United States
(12) Patent Application Publication
(10) Pub. No.: US 2020/0054697 A1
(43) Pub. Date: Feb. 20, 2020

(54) COMMENSAL BACTERIA AS NOVEL TREATMENT FOR DRY EYE AND SJOGREN SYNDROME

(71) Applicant: Baylor College of Medicine, Houston, TX (US)

(72) Inventors: Cintia S. De Paiva, Houston, TX (US); Stephen C. Pflugfelder, Houston, TX (US); Robert Allen Britton, Houston, TX (US)

(21) Appl. No.: 16/604,013

(22) PCT Filed: Apr. 11, 2018
(86) PCT No.: PCT/US2018/027063

§ 371 Date: Oct. 9, 2019

Related U.S. Application Data
(60) Provisional application No. 62/486,307, filed on Apr. 17, 2017.

(51) Int. Cl. A61K 35/747; A61P 27/04; A61P 37/06

(52) U.S. Cl. CPC A61K 35/747; A61P 37/06; A61P 27/04

(57) ABSTRACT

Embodiments of the disclosure encompass methods of treating or preventing an autoimmune disease in an individual. In particular cases, methods comprise administering for delivery to an individual a composition of microbiota. In certain cases, the composition comprises a population of one or more microbiota capable of producing one or more short-chain fatty acids.

This application claims priority to U.S. Provisional Patent Application Ser. No. 62/486,307, filed Apr. 17, 2017, which is incorporated by reference herein in its entirety.

### TECHNICAL FIELD

Embodiments of this invention generally relate at least to cell biology, molecular biology, bacteriology, medicine, gastroenterology, and microbiology.

### BACKGROUND

Microbiota is the ecological community of commensal, symbiotic and pathogenic microorganisms that literally share our body space. There are trillions of microbes in the body which account for about 1-3% of the total body mass. They help digest food, metabolism, and contribute to the maturation of the immune system and homeostasis. (Ruff et al., 2015) Microbiota especially in the gut plays an important role in barrier against pathogens, maintenance of intestinal homeostasis and modulation of the host immune system. (Hooper et al., 2012) Microbial balance and integrity are important for good health. Microbiota composition is influenced by environmental factors such as diet, antibiotic therapy and environmental exposure to microorganisms. A loss of balance (dysbiosis) can trigger digestive dysfunctions, allergies in children and chronic conditions including obesity and inflammatory diseases (Burcelin et al., 2012).

Sjögren syndrome (SS) is an autoimmune disorder that affects exocrine glands such as salivary and lacrimal glands (LG) with lymphocytic infiltration leading to dry eye and mouth. These glands have significant infiltration that results in apoptosis and acinar loss (Kong et al., 1998, Ishimaru et al., 1999, Kimura-Shimmyo et al., 2002, Zoukhri, 2010). The infiltrating cells are a mix of T-cells, B-cells, dendritic cells and natural killer cells (NK) (Christodoulou et al., 2010).

The present disclosure satisfies a long-felt need in the art to provide suitable therapies for autoimmune disorders including SS.

### BRIEF SUMMARY

The present disclosure provides methods and compositions for treating or preventing at least one autoimmune disease in an individual. In specific embodiments, the disclosure concerns methods that include administering for delivery to the gastrointestinal tract of the individual a composition of microbiota, wherein the composition comprises a population of one or more microbiota capable of producing one or more short-chain fatty acids. In some aspects, one or more butyrate-producing bacteria are utilized for the treatment or prevention of an autoimmune disorder, such as SS, and may be used for dry eye; in particular aspects the bacteria are indirectly or directly delivered to the gastrointestinal tract at any point. In certain cases, Lactobacillus reuteri is utilized for treatment of dry eye or SS. In particular embodiments, fecal transplants are utilized for treatment of autoimmune disease such as SS or for dry eye of any kind.

In particular embodiments, the disclosure provides a composition of one or more microbiota which comprises, consists of, or consists essentially of Faecalibacterium prausnitzii, Anaerostipes, Eubacterium, Roseburia, Lactobacillus reuteri, Bacteroides, Blautia, Coprococcus, or combinations thereof. In specific cases, there is a composition of one or more microbiota which comprises, consists of, or consists essentially of Acetanaerobacterium, Acetivibrio, Akkermansia, Alicyclobacillus, Alkaliphilus, Anaerofustis, Anaerosporobacter, Anaerostipes, Anaerotruncus, Anoxybacillus, Bacillus, Bacteroides, Blautia, Brachyspira, Brevibacillus, Bryantella, Bulleidia, Butyricicoccus, Butyrivibrio, Catenibacterium, Chlamydiales, Clostridiaceae, Clostridiales, Clostridium, Collinsella, Coprobacillus, Coprococcus, Coxiella, Deferribacteres, Desulfitobacterium, Desulfotomaculum, Dorea, Eggerthella, Erysipelothrix, Erysipelotrichaceae, Ethanoligenens, Eubacterium, Faecalibacterium, Filifactor, Flavonifractor, Flexistipes, Fulvimonas, Fusobacterium, Gemmiger, Geobacillus, Gloeobacter, Holdemania, Hydrogenoanaerobacterium, Kocuria, Lachnobacterium, Lachnospira, Lachnospiraceae, Lactobacillus, Lactonifactor, Leptospira, Lutispora, Lysinibacillus, Mollicutes, Moorella, Nocardia, Oscillibacter, Oscillospira, Paenibacillus, Papillibacter, Pseudoflavonifractor, Robinsoniella, Roseburia, Ruminococcaceae, Ruminococcus, Saccharomonospora, Sarcina, Solobacterium, Sporobacter, Sporolactobacillus, Streptomyces, Subdoligranulum, Sutterella, Syntrophococcus, Thermoanaerobacter, Thermobifida, Turicibacter, Acetonema, Amphibacillus, Ammonifex, Anaerobacter, Caldicellulosiruptor, Caloramator, Candidatus, Carboxydibrachium, Carboxydothermus, Cohnella, Dendrosporobacter Desulfitobacterium, Desulfosporosinus, Halobacteroides, Heliobacterium, Heliophilum, Heliorestis, Lachnoanaerobaculum, Oceanobacillus, Orenia (S.), Oxalophagus, Oxobacter, Pelospora, Pelotomaculum, Propionispora, Sporohalobacter, Sporomusa, Sporosarcina, Sporotomaculum, Symbiobacterium, Syntrophobotulus, Syntrophospora, Terribacillus, Thermosinus or combinations thereof. In a particular embodiment the composition is comprised of microbiota derived from a fecal sample of a healthy human donor.

One aspect of the invention provides a composition of microbiota wherein the composition of microbiota may comprise, consist, or consist essentially of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 50, or no more than 100 type(s) of microbiota. Additionally, the invention provides a composition of microbiota wherein the composition of microbiota may comprise, consist, or consist essentially of between 1 and 100, 1 and 50, or 1 and 20; or 1 and 10, 2 and 10, 3 and 10, 4 and 10, 5 and 10, 6 and 10, 7 and 10, 8 and 10, or 9 and 10; or 1 and 9, 2 and 9, 3 and 9, 4 and 9, 5 and 9, 6 and 9, 7 and 9, or 8 and 9; or 1 and 8, 2 and 8, 3 and 8, 4 and 8, 5 and 8, 6 and 8, or 7 and 8; or 1 and 7, 2 and 7, 3 and 7, 4 and 7, 5 and 7, or 6 and 7; or 1 and 6, 2 and 6, 3 and 6, 4 and 6, or 5 and 6; or 1 and 5, 2 and 5, 3 and 5, or 4 and 5; or 1 and 4, 2 and 4, or 3 and 4; or 1 and 3, or 2 and 3; or 1 and 2; or 1 type(s) of microbiota. Furthermore, the invention provides a composition of microbiota wherein the composition comprises, consists of, or consists essentially of one type of microbiota present in amounts at least 2, 5, 10, 25, 50, 75, 100 or more than 100 times greater than any other type of microbiota present in the composition.

In some cases the disclosure provides a composition of microbiota wherein in the composition the majority of microbiota comprises, consists of, or consists essentially of Lactobacillus reuteri, Bacteroides, Blautia, and/or Coprococcus. Another aspect of the invention provides a composition of microbiota wherein in the composition the majority of microbiota comprises, consists of, or consists essentially of two or more of Lactobacillus reuteri, Bacteroides, Blautia, or Coprococcus. Yet another aspect of the disclosure provides a composition of microbiota wherein in the composition the majority of microbiota comprises, consists of, or consists essentially of three or more of Lactobacillus reuteri, Bacteroides, Blautia, or Coprococcus.

In some cases the disclosure provides a composition of microbiota wherein in the composition Lactobacillus reuteri is at least 25, 30, 35, 40, 45, 50, 55, 60, 65, 70, 75, 80, 85, 90, 95, 96, 97, 98, 99, or greater than 99% of the microbiota in the composition. In some cases the invention provides a composition of microbiota wherein in the composition Bacteroides is at least 25, 30, 35, 40, 45, 50, 55, 60, 65, 70, 75, 80, 85, 90, 95, 96, 97, 98, 99, or greater than 99% of the microbiota in the composition. In some cases the invention provides a composition of microbiota wherein in the composition Blautia is at least 25, 30, 35, 40, 45, 50, 55, 60, 65, 70, 75, 80, 85, 90, 95, 96, 97, 98, 99, or greater than 99% of the microbiota in the composition. In some cases the invention provides a composition of microbiota wherein in the composition Coprococcus is at least 25, 30, 35, 40, 45, 50, 55, 60, 65, 70, 75, 80, 85, 90, 95, 96, 97, 98, 99, or greater than 99% of the microbiota in the composition.

One aspect of the disclosure provides a composition of microbiota wherein the relative presence of microbiota in the composition is expressed as a ratio of a first type of microbiota to a second type of microbiota comprising, consisting of, or consisting essentially of 1:1, 1:2, 1:3, 1:4, 1:5, 1:6, 1:7, 1:8, 1:9, 1:10, 1:15, 1:20, 1:25; 1:50; 1:75, 1:100, 1:200, 1:500, 1:1000, 1:10,000, 1:100,000 or greater than 1:100,000. Another aspect of the invention provides a composition of microbiota wherein the concentration of a given microbiota or the concentration of the aggregate composition comprises 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ viable microbiota per gram of composition.

One aspect of the disclosure provides a method for treating one or more autoimmune disease(s) comprising: Sjögren syndrome, Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Anti-TBM nephritis, Antiphospholipid syndrome, arthofibrosis, atrial fibrosis, autoimmune angioedema, autoimmune aplastic anemia, autoimmune dusautonomia, autoimmune hepatitis, autoimmune hyperlipidemia, autoimmune immunodeficiency, autoimmune inner ear disease, autoimmune myocarditis, autoimmune oophoritis, autoimmune pancreatitis, autoimmune retinopathy, autoimmune thrombocytopenic purpura, autoimmune thyroid disease, autoimmune urticaria, axonal and neuronal neuropathies, Balo disease, Behcet's disease, benign mucosal pemphigold, Bullous pemphigold, cardiomyopathy, Castleman disease, Celiac Disease, Chagas disease, chronic fatigue syndrome, chronic inflammatory demyelinating polyneuropathy, chronic Lyme disease, chronic recurrent multifocal osteomyelitis, Churg-Strauss syndrome, cicatricial pemphigold, cirrhosis, Cogans syndrome, cold agglutinin disease, congenital heart block, Coxsackle myocarditis, CREST disease, Crohn's disease, Cystic Fibrosis, essential mixed cryoglobulinemia, deficiency of the interleukin-1 receptor antagonist, demyelinating neuropathies, dermatitis herpetiformis, dermatomyosis, Devic's disease, discoid lupus, Dressler's syndrome, Dupuytren's contracture, endometriosis, endomyocardial fibrosis, eosinophilic esophagitis, eosinophilic facsciitis, erythema nodosum, experimental allergic encephalomyelitis, Evans syndrome, Familial Mediterranean Fever, fibromyalgia, fibrosing alveolitis, giant cell arteritis, giant cell myocarditis, glomerulonephritis, Goodpasture's syndrome, Graft-versus-host disease (GVHD), granulomatosus with polyanglitis, Graves' disease, Guillain-Bare syndrome, Hashimoto's encephalitis, Hashimoto's thyroiditis, hemolytic anemia, Henoch-Schonlein purpura, hepatitis, herpes gestationis, hypogammaglobulinemia, idiopathic thrombocytopenic purpura, IgA nephropathy, IgG4-related sclerosing disease, immunoregulatory lipoproteins, inclusion body myositis, inflammatory bowel disorders, interstitial cystitis, juvenile arthritis, juvenile myositis, Kawasaki syndrome, keloid, Lambert-Eaton syndrome, leukocytoclastic vasculitis, lichen planus, lichen sclerosus, ligneous conjunctivitis, linear IgA disease, mediastinal fibrosis, Meniere's disease, microscopic polyanglitis, mixed connective tissue disease, Mooren's ulcer, Mucha-Hamermann disease, Multiple Sclerosis (MS), Myasthenia gravis, myelofibrosis, Myositis, narcolepsy, Neonatal Onset Multisystem Inflammatory Disease, nephrogenic systemic fibrosis, neutropenia, nonalcoholic fatty liver disease, nonalcoholic steatohepatitis (NASH), ocular-cicatricial pemphigold, optic neuritis, palindromic rheumatism, Pediatric Autoimmune Neuropsychiatric Disorders Associated with Streptococcus (PANDAS), paraneoplastic cerebellar degeneration, paroxysmal nocturnal nemoglobinuria, Parry Romberg syndrome, Parsonnage-Turner syndrome, Pars planitis, Pemphigus, Peripheral neuropathy, perivenous encephalomyelitis, pernicious anemia, Peyronie's disease, POEMS syndrome, polyarteritis nodosa, progressive massive fibrosis, Tumor Necrosis Factor Receptor-associated Periodic Syndrome, Type I autoimmune polyglandular syndrome, Type II autoimmune polyglandular syndrome, Type III autoimmune polyglandular syndrome, polymyalgia rhematica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, sperm and testicular autoimmunity, stiff person syndrome, subacute bacterial endocarditis, Susac's syndrome, sympathetic ophthalmia, systemic lupus erythematosus (SLE), Takayasu's arthritis, temporal arteritis, thrombocytopenic purpura, Tolosa-Hunt syndrome, transverse myelitis, Type 1 diabetes, ulcerative colitis, undifferentiated connective tissue disease, uveitis, vasculitis, vesiculobullous dermatosis, and Vitiligo.

In specific cases, the microbiota of the composition comprise one or more bacteria capable of producing one or more short-chain fatty acid(s) selected from the group consisting of butyrate, acetate, propionate, valerate, and combinations thereof. In certain cases, the one or more bacteria in the composition is capable of producing at least 1 mM, or at least 2 mM, or at least 3 mM, or at least 4 mM, or at least 5 mM, or at least 6 mM, or at least 7 mM, or at least 8 mM, or at least 9 mM, or at least 10 mM of short-chain fatty acid per gram of composition.

In particular embodiments, the composition is administered to a subject by a method suitable for depositing in the gastrointestinal tract, preferably the colon, of a subject (e.g., human, mammal, animal, etc.). Examples of routes of administration include rectal administration by colonoscopy, suppository, enema, upper endoscopy, upper push enteroscopy. Additionally, intubation through the nose or the mouth by nasogastric tube, nasoenteric tube, or nasal jejunal tube may be utilized. Oral administration by a solid such as a pill, tablet, a suspension, a gel, a geltab, a semisolid, a tablet, a sachet, a lozenge or a capsule or microcapsule, or as an enteral formulation, or re-formulated for final delivery as a liquid, a suspension, a gel, a geltab, a semisolid, a tablet, a sachet, a lozenge or a capsule, or as an enteral formulation may be utilized as well.

### BRIEF DESCRIPTION OF THE DRAWINGS

FIGS. 1A-1H. Sjögren-like Lacrimal Keratoconjunctivitis on GF mice. Conventionally housed mice with complex flora (CON) were compared to germ-free (GF) mice at 8 weeks of age. Both sexes were pooled. (FIG. 1A) Corneal Oregon-Green dextran fluorescence intensity score. Bar graphs show mean±SEM of two independent experiments with four-five animals per experiment (final n=8-10 animals, mixed sex). (FIG. 1B) Representative pictures of the corneas stained with Oregon-Green dextran (OGD). (FIG. 1C) Number of PAS conjunctival goblet cells counted in paraffin-embedded sections expressed as number per millimeter. Bar graphs show mean±SEM of two independent experiments with three animals per group, yielding a final sample of six right eyes for each group). (FIG. 1D) Representative images of conjunctiva sections stained with PAS (purple cells) used to generate the bar graph in C. (FIG. 1E) Total LG infiltration measured in H&E stained sections as shown in F (n=6 right LG). (FIG. 1F) Representative pictures of haematoxylin and eosin (H&E)-stained sections of LG. Black quadrants insets are high magnification of while dotted square. (FIG. 1G) Flow cytometric analysis of LG. Right and left extraorbital LGs from one mouse per group were excised and pooled into a single tube, yielding a final sample of 12 individual LG samples divided into two independent experiments with six samples per experiment. Bar graphs show mean±SEM. (FIG. 1H) Tear EGF concentrations were measured by enzyme linked immunosorbent assay. Tear washings from both right and left eyes from one mouse per group were collected and pooled into a single tube, yielding a final sample of 12 individual samples per group and divided into three independent experiments with four samples per experiment).

FIGS. 2A-2E. CD4+ T Cells from GF Mice transfer Sjögren-like Lacrimal Keratoconjunctivitis in Immunodeficient RAG1KO mice. CD4+ T cells were isolated from spleens and cervical lymph nodes (CLN) and adoptively transferred into RAG1KO mice. Disease severity parameters were evaluated 5 weeks later. CD4+ T cells were isolated from spleens and cervical lymph nodes (CLN) from either 8-week old germ-free C57BL/6 mice (GF KO) or conventional flora (CF KO) mice and adoptively transferred (AT) i.p. into RAG1KO recipients (AT). Ocular and lacrimal gland phenotype in RAG1KO recipients was investigated 5 weeks post-transfer. Only female mice were used. A separate group of GF mice received oral gavage (OG) of fecal material at 4 weeks of age. (FIG. 2A) Corneal Oregon-Green Dextran fluorescence intensity score. Bar graphs show mean±SEM of two independent experiments with four animals per experiment (final n=8 animals). (FIG. 2B) Number of PAS+ conjunctival goblet cells counted in paraffin-embedded sections expressed as number per millimeter. Bar graphs show means±SEM of two independent experiments with three animals per group, yielding a final sample of six right eyes for each group). Parametric t-test statistical tests were used to make comparisons between groups. (FIG. 2C) LG percentage infiltration measured in H&E stained sections. N=5. (FIG. 2D) Flow cytometric analysis of intracellular staining of LG and CLN of adoptive transfer recipients. Right and left extraorbital LGs from one mouse per group were excised and pooled into a single tube, yielding a final sample of six individual LG samples divided into two independent experiments with three samples per experiment. Bar graphs show means±SEM. Parametric t-tests were used to make comparisons between groups. (FIG. 2E) Gene expression analysis in LG lysates of GF and CON mice. Bar graphs show means±SD of six samples per group/age. Parametric t-tests were used to make comparisons between groups.

FIGS. 3A-3D. Reconstitution of GF Mice with Commensal Bacteria Reverses the Dry Eye Phenotype. 4-week old female GF mice were colonized with a fecal slurry from normal mice (a pool of three mice) by intragastric gavage and sacrificed at 8 weeks of age (GF+Fecal gavage, GF+FG). (FIG. 3A) Corneal Oregon-Green dextran fluorescence intensity score. Bar graphs show mean±SEM of two independent experiments with four animals per experiment (final n=8 animals, female sex). (FIG. 3B) Number of PAS conjunctival goblet cells counted in paraffin-embedded sections expressed as number per millimeter. Bar graphs show mean±SEM of two independent experiments with three-four animals per group, yielding a final sample of seven right eyes for each group). (FIG. 3C) Representative images of conjunctiva sections stained with PAS used to generate the bar graph in B. (FIG. 3D) Fecal transplant during desiccating stress (DS) rescues goblet cells. Female CON C57BL/6 mice were left untreated (naïve mice) or received a cocktail of antibiotics (ABX) for 7 days. On the morning of the 8th day, mice were switched to normal water and subjected to desiccating stress (DS) for 10 days (DS10) and randomized to receive either PBS or oral gavage of fecal material or were left nonstressed. Mice under DS were sacrificed after 10 days and number of PAS+ cells in the conjunctiva was determined. N=5 animals/group.

FIGS. 4A-4H. GF environment worsens SS in the CD25KO mice. Conventionally housed CD25KO (KO) mice with complex flora (CON) were compared to germ-free (GF) KO mice at 4 and 8 weeks of age. Both sexes were used. (FIGS. 4A-4B) Representative pictures of the corneal permeability (FIG. 4A) and accumulative data (FIG. 4B). Corneas were stained with fluorescent Oregon-Green dextran (OGD) dye and OGD intensity score was calculated in the 2-mm central cornea by two masked investigators. Bar graphs show means±SEM of two independent experiments with four-five animals per experiment (final n=8-10 animals). Parametric t-test were used to make comparisons between groups. (FIG. 4C) Number of PAS+ conjunctival goblet cells counted in paraffin-embedded sections expressed as number per millimeter. Bar graphs show means±SEM of two independent experiments with three animals per group, yielding a final sample of six right eyes for each group. Parametric t-test statistical tests were used to make comparisons between groups. (FIG. 4D) Total LG infiltration measured in H&E stained sections as shown in F (n=6 right LG). Nonparametric Mann-Whitney U statistical tests were used to make comparisons of inflammation scores (final n=8-10 animals). (FIG. 4E) Representative pictures of haematoxylin and eosin (H&E)-stained sections of LG. Black quadrants insets are high magnification of while dotted square. 10× magnification. (FIG. 4F) Flow cytometric analysis of LG stained for CD4, CD8 and B220 at 4 and 8 weeks of age. Bar graphs show means±SD of six samples per group/age. Parametric t-tests were used to make comparisons between groups. (FIG. 4G) Gene expression analysis in LG lysates of GF and CON mice. Bar graphs show means±SD of six samples per group/age. Parametric t-tests were used to make comparisons between groups. (FIG. 411) Flow cytometric analysis of intracellular staining of LG and CLN of CON and GF mice at 8 weeks of age. Right and left extraorbital LGs from one mouse were excised and pooled into a single tube, yielding a final sample of twelve individual LG samples divided into two independent experiments with six samples per experiment. Bar graphs show means±SEM. Parametric t-tests were used to make comparisons between groups.

FIGS. 5A-5F. Adoptive transfer recipients of GF KO CD4+ T cells develop SS-like disease. CD4+ T cells were isolated from spleens and cervical lymph nodes (CLN) from either germ-free CD25KO (GF KO) or conventional CD25KO (CON KO) mice and adoptively transferred (AT) i.p. into RAG1KO recipients (AT□RAG). Ocular and lacrimal gland phenotype in RAG1KO recipients was investigated 5 weeks post-transfer. (FIG. 5A) Corneal permeability measured as an uptake of fluorescent Oregon-Green dextran (OGD) dye. Bar graphs show means±SEM of two independent experiments with four-five animals per experiment (final n=8-10 animals). Parametric t-tests were used to make comparisons between groups. (FIG. 5B) Number of PAS+ conjunctival goblet cells counted in paraffin-embedded sections expressed as number per millimeter. Bar graphs show means±SEM of two independent experiments with three animals per group, yielding a final sample of six right eyes for each group. Parametric t-tests were used to make comparisons between groups. (FIG. 5C) Inflammation scores of LG pathology of donor and AT recipients. Nonparametric Mann-Whitney U statistical tests were used to make comparisons of inflammation scores (final n=8-10 animals). (FIG. 5D) Representative pictures of haematoxylin and eosin (H&E)-stained sections of LG in adoptive transfer RAG1KO recipients. 20× magnification, scale bar=50 (FIG. 5E) Flow cytometric analysis of intracellular staining of LG and CLN of adoptive transfer recipients. Right and left extraorbital LGs from one mouse per group were excised and pooled into a single tube, yielding a final sample of six individual LG samples divided into two independent experiments with three samples per experiment. Bar graphs show means±SEM. Parametric t-tests were used to make comparisons between groups. (FIG. 5F) Gene expression analysis in LG lysates of GF and CON KO adoptive transfer recipients. (AT->RAG). Bar graphs show means±SD (n=four to five LG/group).

FIGS. 6A-6B. Conventionalization of CD25KO ameliorates the autoimmune phenotype. Germ-free CD25KO (GF KO) received an oral gavage (OG) of fecal slurry at 4 weeks of age and were sacrificed at 8 weeks of age. CD4+ T cells were isolated from spleens and cervical lymph nodes (CLN) and adoptively transferred (AT) into RAG1KO (AT→RAG) recipients. Ocular and lacrimal gland phenotype was investigated in both donor mice and RAG1KO recipients. (FIGS. 6A-6D) Donor mice phenotype and adoptive recipients after fecal transplant (FIG. 6A) Corneal permeability measured as an uptake of fluorescent Oregon-Green dextran (OGD) dye. Bar graphs show means±SEM of three independent experiments (final n=eighteen to twenty-one animals). Parametric t-tests were used to make comparisons between groups. (FIG. 6B) Number of PAS⁺ conjunctival goblet cells counted in paraffin-embedded sections. Bar graphs show means±SEM of two independent experiments, final n=five right eyes for each group. Parametric t-tests were used to make comparisons between groups. (FIG. 6C) Inflammation scores of lacrimal gland pathology of donor mice. Bar graphs show average of two independent experiments (final n=ten animals). Nonparametric Mann-Whitney U statistical tests were used to make comparisons of inflammation scores. (FIG. 6D) Representative pictures of haematoxylin and eosin (H&E)-stained sections of lacrimal gland in donor mice. (FIG. 6E) Inflammation scores of lacrimal gland pathology of AT recipients. Bar graphs show average of two independent experiments (final n=ten animals). Nonparametric Mann-Whitney U statistical tests were used to make comparisons of inflammation scores. (FIG. 6F) Flow cytometric analysis of cervical lymph nodes and lacrimal gland of adoptive transfer RAG1KO recipients. Bar graphs show means±SEM of two independent experiments (final n=seven to eleven animals/group). Parametric t-tests were used to make comparisons between groups. *P<0.05; **P<0.01; ***P<0.001; ****P<0.0001

FIGS. 7A-7C. Conventional (CON) wild-type (WT) and CD25KO mice were subjected to a cocktail of oral antibiotics for 4 weeks starting at 4 weeks of age and compared to CON KO at 8 weeks of age that drank normal water. (FIG. 7A) Representative pictures of haematoxylin and eosin (H&E)-stained sections of LG. Black arrows indicate collapsed acini. 20× magnification, scale bar=100 μm. (FIG. 7B) Inflammation scores of LG pathology. Nonparametric Mann-Whitney U statistical tests were used to make comparisons of inflammation scores (n=five animals). (FIG. 7C) Gene expression analysis in LG and conjunctival (CJ) lysates. Bar graphs show means±SEM of five samples per group. Parametric t-tests were used to make comparisons between groups.

FIG. 8. Lower levels of short-chain fatty acids (SCFAs) in stools of SS patients measured by HLPC (N=3/group).

FIGS. 9A-9B. Butyrate producing (BP) bacteria gavage in GF mice improves goblet cell density (FIG. 9A) and corneal staining (FIG. 9B) while Enterococcus (Ent) does not. N=5/group (FIGS. 9A & 9B).

### DETAILED DESCRIPTION OF THE INVENTION

### I. Definitions

In keeping with long-standing patent law convention, the words “a” and “an” when used in the present specification in concert with the word comprising, including the claims, denote “one or more.” Some embodiments of the disclosure may consist of or consist essentially of one or more elements, method steps, and/or methods of the disclosure. It is contemplated that any method or composition described herein can be implemented with respect to any other method or composition described herein.

As used herein, the term “about” or “approximately” refers to a quantity, level, value, number, frequency, percentage, dimension, size, amount, weight or length that varies by as much as 30, 25, 20, 25, 10, 9, 8, 7, 6, 5, 4, 3, 2 or 1% to a reference quantity, level, value, number, frequency, percentage, dimension, size, amount, weight or length. In particular embodiments, the terms “about” or “approximately” when preceding a numerical value indicates the value plus or minus a range of 15%, 10%, 5%, or 1%.

Throughout this specification, unless the context requires otherwise, the words “comprise”, “comprises” and “comprising” will be understood to imply the inclusion of a stated step or element or group of steps or elements but not the exclusion of any other step or element or group of steps or elements. By “consisting of” is meant including, and limited to, whatever follows the phrase “consisting of.” Thus, the phrase “consisting of” indicates that the listed elements are required or mandatory, and that no other elements may be present. By “consisting essentially of” is meant including any elements listed after the phrase, and limited to other elements that do not interfere with or contribute to the activity or action specified in the disclosure for the listed elements. Thus, the phrase “consisting essentially of” indicates that the listed elements are required or mandatory, but that no other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, a “type” or more than one “types” of microbiota may be differentiated at the genus level, the species level, the sub-species level, the strain level or by any other taxonomic method, as described herein and otherwise known in the art.

As used herein, a “fecal sample” refers to a solid waste product of digested food and includes feces or bowel washes, as examples.

As used herein, “isolated” and “isolation” encompasses a microbe or other entity or substance that has been (1) separated from at least some of the components with which it was associated when initially produced (whether in nature or in an experimental setting), and/or (2) produced, prepared, purified, and/or manufactured by the hand of man.

A used herein, a “non-natural” composition encompasses a microbe or other entity or substance that has been (1) separated from at least some of the components with which it was associated when initially produced (whether in nature or in an experimental setting), and/or (2) produced, prepared, purified, and/or manufactured by the hand of man. Non-natural compostions of microbiota include, for example, those microbiota that are cultured, even if such cultures are not monocultures. Non-natural compostions of microbiota may be separated from at least about 10%, about 20%, about 30%, about 40%, about 50%, about 60%, about 70%, about 80%, about 90%, or more of the other components with which they were initially associated. In some embodiments, non-natural compostions of microbiota are more than about 80%, about 85%, about 90%, about 91%, about 92%, about 93%, about 94%, about 95%, about 96%, about 97%, about 98%, about 99%, or more than about 99% pure.

As used herein, a substance is “pure” if it is substantially free of other components. The terms “purify,” “purifying” and “purified” refer to a population of one or more microbiota or other material that has been separated from at least some of the components with which it was associated either when initially produced or generated (e.g., whether in nature or in an experimental setting), or during any time after its initial production. In some embodiments, a purified population of one or more microbiota are more than about 80%, about 85%, about 90%, about 91%, about 92%, about 93%, about 94%, about 95%, about 96%, about 97%, about 98%, about 99%, or more than about 99% pure. In the instance of compositions comprising populations of one or more microbiota provided herein, the one or more microbiota types present in the composition may be independently purified from the material or environment containing the microbiota type. In other embodiments, compositions contain a defined mixture of isolated microbiota.

For example, in some embodiments, the composition comprises or contains no more than 100 bacterial species. For example, in some embodiments, the probiotic composition contains no more than 75 bacterial species. In other embodiments, the probiotic composition contains no more than 100 bacterial species, e.g., no more than 40 bacterial species, no more than 30 bacterial species, no more than 25 bacterial species, no more than 20 bacterial species, no more than 15 bacterial species, no more than 10 bacterial species, etc. In other embodiments, the probiotic composition contains no more than 10 bacterial species, e.g., 10 bacterial species, 9 bacterial species, 8 bacterial species, 7 bacterial species, 6 bacterial species, 5 bacterial species, 4 bacterial species, 3 bacterial species, 2 bacterial species, or 1 bacterial species.

“Microbiota” refers to the community of microorganisms that inhabit (sustainably or transiently) in and/or on a subject, (e.g., a mammal such as a human), including, but not limited to, eukaryotes (e.g., protozoa), archaea, bacteria, and viruses (including bacterial viruses, i.e., a phage).

“Microbiome” refers to the genetic content of the communities of microbes that live in and on the human body, both sustainably and transiently, including eukaryotes, archaea, bacteria, and viruses (including bacterial viruses (i.e., phage)), wherein “genetic content” includes genomic DNA, RNA such as ribosomal RNA, the epigenome, plasmids, and all other types of genetic information.

The “colonization” or “recolonization” of a host organism includes the non-transitory residence of a bacterium or other microscopic organism.

As used herein “preventing” or “prevention” refers to any methodology where the disease state does not occur due to the actions of the methodology (such as, for example, administration of microbiota as described herein). In one aspect, it is understood that prevention can also mean that the disease is not established to the extent that occurs in untreated controls. For example, there can be a 5, 10, 15, 20, 25, 30, 35, 40, 50, 60, 70, 80, 90, or 100% reduction in the establishment of disease frequency relative to untreated controls. Accordingly, prevention of a disease encompasses a reduction in the likelihood that a subject will develop the disease, relative to an untreated subject (e.g. a subject who does not receive microbiota as described herein).

The term “subject” or “individual” refers to any organism or animal subject that is an object of a method or material, including mammals, e.g., humans, laboratory animals (e.g., primates, rats, mice, rabbits), livestock (e.g., cows, sheep, goats, pigs, turkeys, and chickens), household pets (e.g., dogs, cats, and rodents), horses, and transgenic non-human animals. The subject may be suffering from dysbiosis, including, but not limited to, an infection due to a gastrointestinal pathogen or may be at risk of developing or transmitting to others an infection due to a gastrointestinal pathogen. Synonyms used herein include “patient” and “animal.”

“Treatment,” “treat,” or “treating” means a method of reducing the effects of a disease or condition. Treatment can also refer to a method of reducing the disease or condition itself rather than just the symptoms. The treatment can be any reduction from pre-treatment levels and can be but is not limited to the complete ablation of the disease, condition, or the symptoms of the disease or condition. Therefore, in the disclosed methods, treatment” can refer to a 10%, 20%, 30%, 40%, 50%, 60%, 70%, 80%, 90%, or 100% reduction in the severity of an established disease or the disease progression. For example, a disclosed method for reducing the effects of Sjögren syndrome is considered to be a treatment if there is a 10% reduction in one or more symptoms of the disease in a subject with Sjögren syndrome when compared to pre-treatment levels in the same subject or control subjects. Thus, the reduction can be a 10, 20, 30, 40, 50, 60, 70, 80, 90, 100%, or any amount of reduction in between as compared to native or control levels. It is understood and herein contemplated that “treatment” does not necessarily refer to a cure of the disease or condition, but an improvement in the outlook of a disease or condition (e.g., Sjögren syndrome). In specific embodiments, treatment refers to the lessening in severity or extent of at least one symptom and may alternatively or in addition refer to a delay in the onset of at least one symptom.

### II. General Embodiments

Embodiments of the disclosure provide methods and compositions for the treatment or prevention of at least one autoimmune disease in an individual. Embodiments of the disclosure provide methods for administration of at least one composition comprising at least one population of one or more microbiota to an individual having or at risk of autoimmune disease. In some embodiments, methods comprise administering for delivery (directly or indirectly) to the gastrointestinal tract of the individual at least one composition of microbiota, in specific cases wherein the composition comprises a population of one or more microbiota capable of producing one or more short-chain fatty acids (SCFAs). In alternative cases, the microbiota do not produce one or more short-chain fatty acids.

### III. Compositions of Microbiota

In certain embodiments, compositions of microbiota comprise at least a population of one or more microbiota, and optionally further comprise microbiota capable of producing SCFAs.

In one embodiment, the microbiota can be produced by isolation and/or culture, using, for example, the following steps: a) providing fecal material and b) subjecting the material to a culture step and/or a treatment step resulting in purification and/or isolation of preferred microbiota and, optionally, c) formulating the purified population for administration, wherein the purified population is present in the composition in an amount effective to engraft and/or colonize in the gastrointestinal tract in order to treat, prevent or reduce the severity of one or more symptom of an autoimmune disease, e.g. Sjorgren's syndrome (SS), in a mammalian recipient subject to whom the therapeutic composition is administered.

Generally, the composition of a population of one or more microbiota comprise, consist of, or consist essentially of Acetanaerobacterium, Acetivibrio, Akkermansia, Alicyclobacillus, Alkaliphilus, Anaerofustis, Anaerosporobacter, Anaerostipes, Anaerotruncus, Anoxybacillus, Bacillus, Bacteroides, Blautia, Brachyspira, Brevibacillus, Bryantella, Bulleidia, Butyricicoccus, Butyrivibrio, Catenibacterium, Chlamydiales, Clostridiaceae, Clostridiales, Clostridium, Collinsella, Coprobacillus, Coprococcus, Coxiella, Deferribacteres, Desulfitobacterium, Desulfotomaculum, Dorea, Eggerthella, Erysipelothrix, Erysipelotrichaceae, Ethanoligenens, Eubacterium, Faecalibacterium, Filifactor, Flavonifractor, Flexistipes, Fulvimonas, Fusobacterium, Gemmiger, Geobacillus, Gloeobacter, Holdemania, Hydrogenoanaerobacterium, Kocuria, Lachnobacterium, Lachnospira, Lachnospiraceae, Lactobacillus, Lactonifactor, Leptospira, Lutispora, Lysinibacillus, Mollicutes, Moorella, Nocardia, Oscillibacter, Oscillospira, Paenibacillus, Papillibacter, Pseudoflavonifractor, Robinsoniella, Roseburia, Ruminococcaceae, Ruminococcus, Saccharomonospora, Sarcina, Solobacterium, Sporobacter, Sporolactobacillus, Streptomyces, Subdoligranulum, Sutterella, Syntrophococcus, Thermoanaerobacter, Thermobifida, Turicibacter, Acetonema, Amphibacillus, Ammonifex, Anaerobacter, Caldicellulosiruptor, Caloramator, Candidatus, Carboxydibrachium, Carboxydothermus, Cohnella, Dendrosporobacter Desulfitobacterium, Desulfosporosinus, Halobacteroides, Heliobacterium, Heliophilum, Heliorestis, Lachnoanaerobaculum, Oceanobacillus, Orenia (S.), Oxalophagus, Oxobacter, Pelospora, Pelotomaculum, Propionispora, Sporohalobacter, Sporomusa, Sporosarcina, Sporotomaculum, Symbiobacterium, Syntrophobotulus, Syntrophospora, Terribacillus, Thermosinus or combinations thereof.

In certain embodiments, the composition of microbiota may comprise, consist, or consist essentially of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 50, or no more than 100 type(s) of microbiota.

In specific embodiments the composition of microbiota may comprise, consist of, or consist essentially of between 1 and 100, 1 and 50, or 1 and 20; or 1 and 10, 2 and 10, 3 and 10, 4 and 10, 5 and 10, 6 and 10, 7 and 10, 8 and 10, or 9 and 10; or 1 and 9, 2 and 9, 3 and 9, 4 and 9, 5 and 9, 6 and 9, 7 and 9, or 8 and 9; or 1 and 8, 2 and 8, 3 and 8, 4 and 8, 5 and 8, 6 and 8, or 7 and 8; or 1 and 7, 2 and 7, 3 and 7, 4 and 7, 5 and 7, or 6 and 7; or 1 and 6, 2 and 6, 3 and 6, 4 and 6, 5 and 6; 1 and 5, 2 and 5, 3 and 5, 4 and 5; 1 and 4, 2 and 4, 3 and 4; 1 and 3, 2 and 3; 1 and 2; or 1 type(s) of microbiota.

In additional embodiments the composition comprises, consists of, or consists essentially of one type of microbiota present in amounts at least 2, 5, 10, 25, 50, 75, 100 or more than 100 times greater than any other type of microbiota present in the composition.

In one embodiment the majority of microbiota in the composition is Lactobacillus reuteri, Bacteroides, Blautia, or Coprococcus.

In another embodiment the majority of microbiota in the composition is two or more of Lactobacillus reuteri, Bacteroides, Blautia, or Coprococcus.

In yet another embodiment the majority of microbiota in the composition is three or more of Lactobacillus reuteri, Bacteroides, Blautia, or Coprococcus.

In certain embodiments any particular bacteria identified herein is at least 25, 30, 35, 40, 45, 50, 55, 60, 65, 70, 75, 80, 85, 90, 95, 96, 97, 98, 99, or greater than 99% of the microbiota in the composition.

In certain embodiments Lactobacillus reuteri is at least 25, 30, 35, 40, 45, 50, 55, 60, 65, 70, 75, 80, 85, 90, 95, 96, 97, 98, 99, or greater than 99% of the microbiota in the composition.

In specific embodiments Bacteroides is at least 25, 30, 35, 40, 45, 50, 55, 60, 65, 70, 75, 80, 85, 90, 95, 96, 97, 98, 99, or greater than 99% of the microbiota in the composition.

In particular embodiments Blautia is at least 25, 30, 35, 40, 45, 50, 55, 60, 65, 70, 75, 80, 85, 90, 95, 96, 97, 98, 99, or greater than 99% of the microbiota in the composition.

In certain embodiments Coprococcus is at least 25, 30, 35, 40, 45, 50, 55, 60, 65, 70, 75, 80, 85, 90, 95, 96, 97, 98, 99, or greater than 99% of the microbiota in the composition.

In one embodiment the relative presence of microbiota in the composition is expressed as a ratio of a first type of microbiota to a second type of microbiota comprising, consisting of, or essentially consisting of 1:1 or any ratio other than 1:1, such as 1:2, 1:3, 1:4, 1:5, 1:6, 1:7, 1:8, 1:9, 1:10, 1:15, 1:20, 1:25; 1:50; 1:75, 1:100, 1:200, 1:500, 1:1000, 1:10,000, 1:100,000 or greater than 1:100,000.

In certain embodiments, the population of one or more microbiota is provided in an amount effective to treat (including to prevent) a disease, disorder or condition associated autoimmunity, e.g., SS. Such effective amounts may comprise 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵ or greater than 1×10¹⁵ viable microbiota per gram of composition, wherein the composition comprises a population of one or more microbiota.

In specific embodiments, the composition of microbiota is comprised of microbiota isolated from a fecal sample.

In certain embodiments treatment with the composition of microbiota may be effective to reduce the severity of at least one symptom of the SS. Such treatment may be effective to modulate the microbiota diversity present in the mammalian recipient.

In one embodiment, a composition containing or comprising microbiota can treat one or more symptoms of Sjorgren's syndrome in a subject e.g. dry or burning eyes, dry mouth, sore or cracked tongue, dry or burning throat, dry or peeling lips, a change in taste or smell, increased dental decay, joint pain, vaginal and skin dryness, digestive problems, dry nose, and debilitating fatigue.

In other embodiments, a composition containing or comprising microbiota can treat or prevent lacrimal keratoconjunctivitis in a subject.

In one embodiment, a composition containing microbiota can treat or prevent goblet cell loss in a subject. In other embodiments, a composition containing microbiota can treat or prevent corneal barrier disruption in a subject.

In specific embodiments, a composition containing microbiota can reduce pathogenic CD4+ T cell infiltration of the lacrimal gland in a subject.

In specific embodiments, a composition containing microbiota can reduce Th1 cell infiltration of the lacrimal gland in a subject.

In specific embodiments, a composition containing microbiota can reduce the generation of autoreactive CD4+ T cells in a subject.

In certain embodiments, the composition of microbiota is comprised of a population of one or more microbiota capable of producing high levels of SCFAs, e.g. at least 1 mM, or at least 2 mM, or at least 3 mM, or at least 4 mM, or at least 5 mM, or at least 6 mM, or at least 7 mM, or at least 8 mM, or at least 9 mM, or at least 10 mM of SCFAs per gram of composition.

In one embodiment, a population of one or more microbiota capable of producing SCFAs is purified from a population of microbiota grown in laboratory culture.

In certain embodiments, the composition is comprised of a population of one or more microbiota which possesses one or more genetic modification(s) not found in a natural setting, including mutation(s) and/or recombinantly expressed gene(s) made by the hand of man.

In specific embodiments, the genetic modification(s) alter the metabolic activity of the microbiota to increase the production of SCFAs.

In additional embodiments, the genetic modification(s) affect the expression of gene(s) which regulate the flux of carbon into SCFA production, and/or gene(s) which catalyze the production of SCFAs.

In additional embodiments, the genetic modification(s) affect the regulatory elements of gene(s) which regulate the flux of carbon into SCFA production, and/or gene(s) which catalyze the production of SCFAs.

In additional embodiments, the genetic modification(s) affect the enzymatic activity of gene(s) which regulate the flux of carbon into SCFA production, and/or gene(s) which catalyze the production of SCFAs.

In certain embodiments, the genetic modification(s) may enable selection of microbiota with certain traits to be from a population of microbiota using an antibiotic selection strategy.

In another embodiment, a population of one or more microbiota capable of producing SCFAs is isolated from a fecal sample. In other embodiments, a population of one or more microbiota capable of producing SCFAs is obtained commercially, including through a bank or repository of microbiota, for example.

In specific embodiments, compositions contain microbiota which are capable of altering the immune activity of a mammalian subject, herein referred to as immunomodulatory microbiota.

In exemplary embodiments, immunomodulatory microbiota are capable of reducing immune cell invasion in a mammalian subject. Immunomodulatory microbiota can act to alter the immune activity of a subject directly or indirectly. For example, immunomodulatory microbiota can produce metabolites such as immunomodulatory short-chain fatty acids (SCFAs). SCFAs produced by immunomodulatory microbiota can include, e.g., butyrate, acetate, propionate, or valerate, or combinations thereof.

In one embodiment, a composition of microbiota is administered to a subject in an amount effective to increase short chain fatty acid production by one or more microbiota in the gut of a mammalian host.

In one embodiment, immunomodulatory microbiota may alter cytokine expression by host immune cells (e.g., macrophages, B lymphocytes, T lymphocytes, mast cells, peripheral blood mononuclear cells (PBMCs), etc.) or other types of host cells capable of cytokine secretion (e.g., endothelia cells, fibroblasts, stromal cells, etc.). In an exemplary embodiment, composition(s) of microbiota are capable of reducing secretion of one or more pro-inflammatory cytokines by host cells (e.g., host immune cells). For example, microbiota can reduce the production of one or more pro-inflammatory cytokines such as but not limited to IFNγ, IL-1β, IL-12, TNFα, Caspase-3, MHC-II, or combinations thereof.

In other embodiments, a composition containing immunomodulatory microbiota can impact the immune activity of a subject by promoting the differentiation and/or expansion of particular subpopulations of immune cells. For example, immunomodulatory microbiota can increase or decrease the proportion of CD4+ T cells, CD8+ T cells, Th17 cells, or Th1 cells in a subject. The increase or decrease in the proportion of immune cell subpopulations may be systemic.

In one embodiment, a composition containing immunomodulatory microbiota can treat symptoms of Sjorgren's syndrome in a subject. In other embodiments, a composition containing immunomodulatory microbiota can treat or prevent lacrimal keratoconjunctivitis in a subject. In one embodiment, a composition containing immunomodulatory microbiota can treat or prevent goblet cell loss in a subject. In other embodiments, a composition containing immunomodulatory microbiota can treat or prevent corneal barrier disruption in a subject. In specific embodiments, a composition containing immunomodulatory microbiota can reduce pathogenic CD4+ T cell infiltration of the lacrimal gland in a subject. In specific embodiments, a composition containing immunomodulatory microbiota can reduce Th1 cell infiltration of the lacrimal gland in a subject. In specific embodiments, a composition containing immunomodulatory microbiota can reduce the generation of autoreactive CD4+ T cells in a subject.

### IV. Administration of Microbiota

In specific embodiments, there is provided a method for the amelioration, stabilization, treatment and/or prevention of an autoimmune disease(s) comprising administering to an individual in need thereof via a delivery vehicle, formulation, composition, pharmaceutical preparation, product of manufacture, container or device the composition of microbiota described herein.

In certain embodiments exemplary autoimmune diseases include, for example, SS, Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Anti-TBM nephritis, Antiphospholipid syndrome, arthofibrosis, atrial fibrosis, autoimmune angioedema, autoimmune aplastic anemia, autoimmune dusautonomia, autoimmune hepatitis, autoimmune hyperlipidemia, autoimmune immunodeficiency, autoimmune inner ear disease, autoimmune myocarditis, autoimmune oophoritis, autoimmune pancreatitis, autoimmune retinopathy, autoimmune thrombocytopenic purpura, autoimmune thyroid disease, autoimmune urticaria, axonal and neuronal neuropathies, Balo disease, Behcet's disease, benign mucosal pemphigold, Bullous pemphigold, cardiomyopathy, Castleman disease, Celiac Disease, Chagas disease, chronic fatigue syndrome, chronic inflammatory demyelinating polyneuropathy, chronic Lyme disease, chronic recurrent multifocal osteomyelitis, Churg-Strauss syndrome, cicatricial pemphigold, cirrhosis, Cogans syndrome, cold agglutinin disease, congenital heart block, Coxsackle myocarditis, CREST disease, Crohn's disease, Cystic Fibrosis, essential mixed cryoglobulinemia, deficiency of the interleukin-1 receptor antagonist, demyelinating neuropathies, dermatitis herpetiformis, dermatomyosis, Devic's disease, discoid lupus, Dressler's syndrome, Dupuytren's contracture, endometriosis, endomyocardial fibrosis, eosinophilic esophagitis, eosinophilic facsciitis, erythema nodosum, experimental allergic encephalomyelitis, Evans syndrome, Familial Mediterranean Fever, fibromyalgia, fibrosing alveolitis, giant cell arteritis, giant cell myocarditis, glomerulonephritis, Goodpasture's syndrome, Graft-versus-host disease (GVHD), granulomatosus with polyanglitis, Graves' disease, Guillain-Bare syndrome, Hashimoto's encephalitis, Hashimoto's thyroiditis, hemolytic anemia, Henoch-Schonlein purpura, hepatitis, herpes gestationis, hypogammaglobulinemia, idiopathic thrombocytopenic purpura, IgA nephropathy, IgG4-related sclerosing disease, immunoregulatory lipoproteins, inclusion body myositis, inflammatory bowel disorders, interstitial cystitis, juvenile arthritis, juvenile myositis, Kawasaki syndrome, keloid, Lambert-Eaton syndrome, leukocytoclastic vasculitis, lichen planus, lichen sclerosus, ligneous conjunctivitis, linear IgA disease, mediastinal fibrosis, Meniere's disease, microscopic polyanglitis, mixed connective tissue disease, Mooren's ulcer, Mucha-Hamermann disease, Multiple Sclerosis (MS), Myasthenia gravis, myelofibrosis, Myositis, narcolepsy, Neonatal Onset Multisystem Inflammatory Disease, nephrogenic systemic fibrosis, neutropenia, nonalcoholic fatty liver disease, nonalcoholic steatohepatitis (NASH), ocular-cicatricial pemphigold, optic neuritis, palindromic rheumatism, Pediatric Autoimmune Neuropsychiatric Disorders Associated with Streptococcus (PANDAS), paraneoplastic cerebellar degeneration, paroxysmal nocturnal nemoglobinuria, Parry Romberg syndrome, Parsonnage-Turner syndrome, Pars planitis, Pemphigus, Peripheral neuropathy, perivenous encephalomyelitis, pernicious anemia, Peyronie's disease, POEMS syndrome, polyarteritis nodosa, progressive massive fibrosis, Tumor Necrosis Factor Receptor-associated Periodic Syndrome, Type I autoimmune polyglandular syndrome, Type II autoimmune polyglandular syndrome, Type III autoimmune polyglandular syndrome, polymyalgia rhematica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, sperm and testicular autoimmunity, stiff person syndrome, subacute bacterial endocarditis, Susac's syndrome, sympathetic ophthalmia, systemic lupus erythematosus (SLE), Takayasu's arthritis, temporal arteritis, thrombocytopenic purpura, Tolosa-Hunt syndrome, transverse myelitis, Type 1 diabetes, ulcerative colitis, undifferentiated connective tissue disease, uveitis, vasculitis, vesiculobullous dermatosis, and Vitiligo.

The composition of microbiota may be administered to a subject daily, every other day, weekly, bi-weekly, monthly, or multiple times in one day for a predefined amount of time to establish amelioration, stabilization, treatment and/or prevention of an autoimmune disease.

The composition of microbiota may be administered to a subject by a method suitable for depositing in the gastrointestinal tract, preferably the colon, of a subject (e.g., human, mammal, animal, etc.). Examples of routes of administration include rectal administration by colonoscopy, suppository, enema, upper endoscopy, upper push enteroscopy. Additionally, intubation through the nose or the mouth by nasogastric tube, nasoenteric tube, or nasal jejunal tube may be utilized. Oral administration by a solid such as a pill, tablet, a suspension, a gel, a geltab, a semisolid, a tablet, a sachet, a lozenge or a capsule or microcapsule, or as an enteral formulation, or re-formulated for final delivery as a liquid, a suspension, a gel, a geltab, a semisolid, a tablet, a sachet, a lozenge or a capsule, or as an enteral formulation may be utilized as well.

### V. Kits of the Disclosure

Any of the compositions described herein or similar thereto may be comprised in a kit. In a non-limiting example, one or more reagents for use in methods for amplification of nucleic acid may be comprised in a kit. Such reagents may include enzymes, buffers, nucleotides, salts, primers, and so forth. The kit components are provided in suitable container means.

Some components of the kits may be packaged either in aqueous media or in lyophilized form. The container means of the kits will generally include at least one vial, test tube, flask, bottle, syringe or other container means, into which a component may be placed, and preferably, suitably aliquoted. Where there are more than one component in the kit, the kit also will generally contain a second, third or other additional container into which the additional components may be separately placed. However, various combinations of components may be comprised in a vial. The kits of the present invention also will typically include a means for containing the components in close confinement for commercial sale. Such containers may include injection or blow molded plastic containers into which the desired vials are retained.

When the components of the kit are provided in one and/or more liquid solutions, the liquid solution is an aqueous solution, with a sterile aqueous solution being particularly useful. In some cases, the container means may itself be a syringe, pipette, and/or other such like apparatus, or may be a substrate with multiple compartments for a desired reaction.

Some components of the kit may be provided as dried powder(s). When reagents and/or components are provided as a dry powder, the powder can be reconstituted by the addition of a suitable solvent. It is envisioned that the solvent may also be provided in another container means. The kits may also comprise a second container means for containing a sterile acceptable buffer and/or other diluent.

In specific embodiments, reagents and materials include primers for amplifying desired sequences, nucleotides, suitable buffers or buffer reagents, salt, and so forth, and in some cases the reagents include apparatus or reagents for isolation of a particular desired cell(s).

In particular embodiments, there are one or more apparatuses in the kit suitable for extracting one or more samples from an individual. The apparatus may be a syringe, fine needles, scalpel, and so forth.

### EXAMPLES

The following examples are included to demonstrate preferred embodiments of the invention. It should be appreciated by those of skill in the art that the techniques disclosed in the examples that follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Microbiome Recolonization of Germ-Free Mice Reverses the Development of Lacrimal Keratoconjunctivitis

Microbiota is the ecological community of commensal, symbiotic and pathogenic microorganisms that literally share host body space. There are trillions of microbes in the body which account for about 1-3% of the total body mass. Microbiota help digest food, metabolism, and contribute to the maturation of the immune system and homeostasis (Ruff et al., 2015). Microbiota in the gut plays an important role in barrier against pathogens, maintenance of intestinal homeostasis and modulation of the host immune system (Hooper et al., 2012). Microbial balance and integrity are important for good health. Microbiota composition is influenced by environmental factors such as diet, antibiotic therapy and environmental exposure to microorganisms. A loss of balance (dysbiosis) can trigger digestive dysfunctions, allergies in children and chronic conditions including obesity and inflammatory diseases (Burcelin et al., 2012).

The inventors have shown that both in SS patients and in the murine DS model that low intestinal microbial diversity correlates with more severe disease phenotype. (de Paiva et al., 2016) This is in agreement with reports that antibiotic treatment exacerbates mucosal inflammation and that recolonization with commensal bacteria reverts inflammatory changes. (Maslowski et al., 2009) The inventors hypothesized that alterations in the microbiome can alter eye phenotype and worsen dry eye disease, including Sjögren Syndrome.

To test this hypothesis, the inventors investigated three different models of dry eye and Sjögren and performed fecal transplant as a novel therapy. In all three models, acute disruption of the microbiome using a cocktail of antibiotics or using showing germ-free mice, the inventors observed a worsening of dry eye phenotype and generation of autoreactive T cells. When a fecal transplant of total bacterial communities was used, a reverse phenotype with greater improvement was observed, indicating a protective role for commensal bacteria. Also provided is data showing that butyrate-producing bacteria are beneficial to the eye.

Model 1: Germ-Free Mice

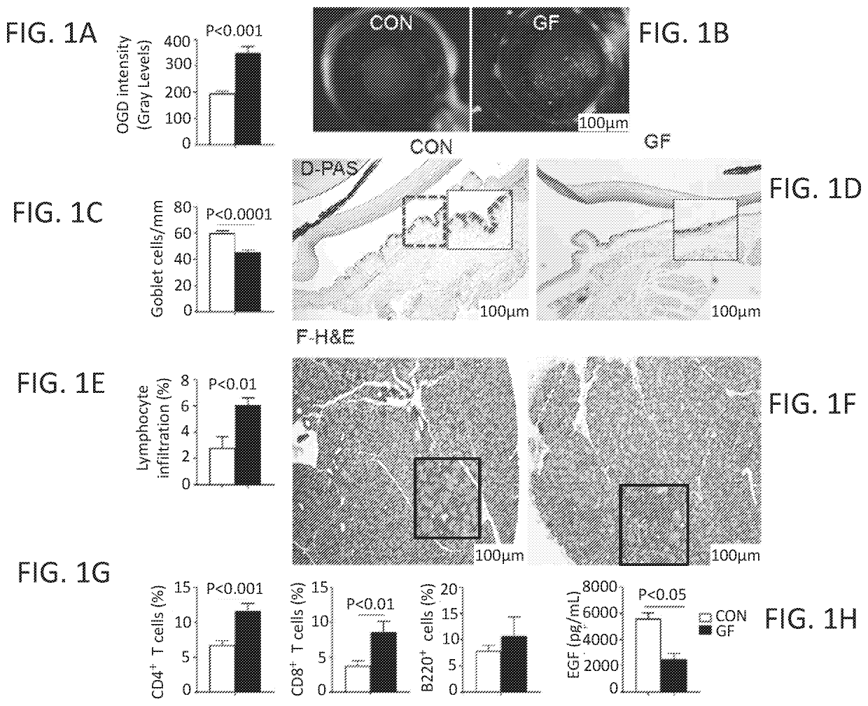

Conventionally housed C57BL/6 with complex flora (CON) animals of both sexes were compared to germ-free (GF) C57BL/6 mice. As seen in FIG. 1A-1B, corneal barrier function studies showed that GF mice have significantly increased permeability to the fluorescent 70-kDa molecule OGD compared with CON mice. Filled goblet cells (GCs) were identified on paraffin embedded conjunctival sections stained with PAS. A significant decrease in GC density from 57.8±1.46 to 44.6±1.33 (cells/mm) in conjunctiva was observed in the GF mice (P<0.0001, FIG. 1C-1D).

To investigate if GF environment would also the affect the other components of the lacrimal gland functional unit, histologic preparations from the extra-orbital LG were prepared. Greater lymphocytic infiltration in GF LG was observed compared to CON mice (FIG. 1E-F); however submandibular glands were not infiltrated in both groups (data not shown). Flow cytometric analyses indicated that the immune cell aggregates in the LG consisted of greater frequency of CD4+, CD8+ T cells, but not B lymphocytes (B220+) in GF compared to CON mice (FIG. 1G). Epidermal growth factor (EGF) is secreted by LGs in humans, is measurable in tears of mice, and tear EGF concentration is significantly lower in murine models of Sjögren syndrome (Yoshino et al., 1996, Pelegrino et al., 2012). The inventors observed that GF mice have significantly lower tear EGF concentration than CON mice (FIG. 1H). These results indicate that 8-week old GF mice have a spontaneously developed Sjögren-like lacrimal keratoconjunctivitis.

GF Alters DC Phenotype and Promotes Generation of Auto-Reactive T Cells.

Previous studies have indicated that CD4+ T cells are a pathogenic T cell population contributing to the onset of Sjögren-like lacrimal keratoconjunctivitis in murine models of SS (McClellan et al., 2014, Niederkorn et al., 2006). In particular, Th1+ cells that secrete IFN-γ have been shown to be prejudicial to the ocular surface and LG (Cha et al., 2004, Pelegrino et al., 2012, de Paiva et al., 2007, Tsubota et al., 1999, Coursey et al., 2016). Next, the inventors investigated if GF environment promotes the generation of autoreactive Th1 CD4+ T cells. To test this hypothesis, adoptive transfer (AT) experiments were performed by isolating CD4+ T cells from female CON and GF nodes and spleens and adoptively transferring these cells into female RAG1KO recipients. Disease parameters were 5 weeks later.

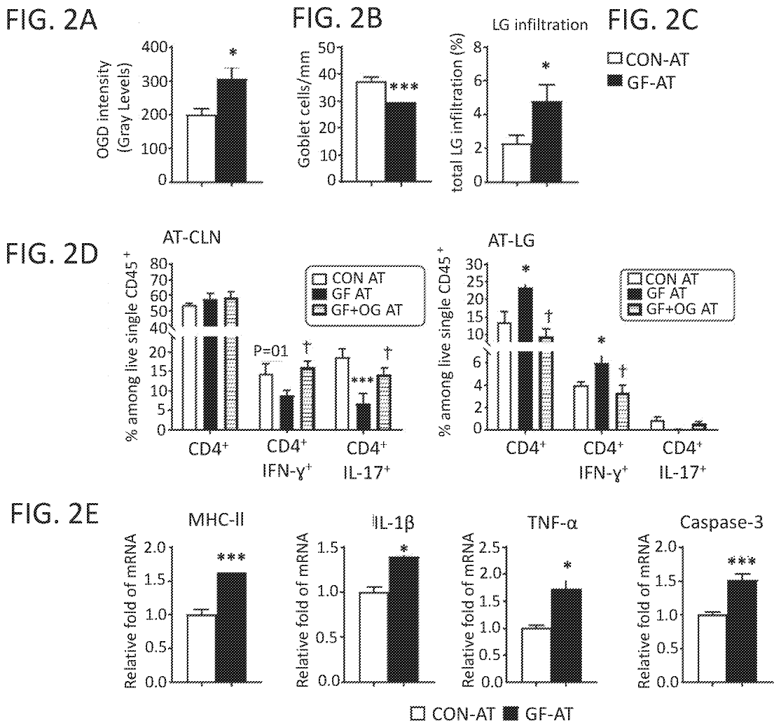

Female RAG1KO GF CD4+ T cell recipients had greater corneal barrier disruption, goblet cell loss and CD4+ T cell infiltration (FIG. 2A-2B), recapitulating the disease phenotype in donor female GF mice (Table 1).

TABLE 1
Summary of Findings to Ocular Surface and Lacrimal Gland with in mice
raised to conventional vivarium (CONV) of in Germ-free (GF) conditions

| | | | | | | | P value | |
|---|---|---|---|---|---|---|---|---|
| | | | | | | | Pattern/ | |
| | | | | | | | sex | Female |
| | | | CON | | GF | | CON | vs. male |
| | Sex/Parameters | | Female | Male | Female | Male | vs. GF | (GF) |
| Ocular | OGD intensity | | 241 ± 75.6 | 228.2 ± 71.5 | 344 ± 118 | 224 ± 147 | ↑, P < 0.001 | F > M |
| surface | (gray levels) | | | | | | female | (P < 001) |
| | Goblet cell | | 56.5 ± 8.8 | 53.2 ± 9.5 | 46.2 ± 7.4 | 43.0 ± 7.0 | ↓, P < 0.001 | |
| | (cells/mm) | | | | | | (both sexes) | |
| | EGF (pg/mL) | | 4282 ± 1288 | 6667 ± 1787 | 2250 ± 750 | 2257 ± 167 | ↓, P < 0.05 | |
| | | | | | | | (both sexes) | |
| LG | Total LG infiltration | | 3.5 ± 3.0 | 1.38 ± 0.4 | 6.36 ± 1.7 | 4.07 ± 2.2 | ↑, P < 0.05 | F > M |
| | score histology (%) | | | | | | (both sexes) | (P < 0.05) |
| | CD4+ T safe (%) | | 6 ± 2.8 | 7.5 ± 3.7 | 10.9 ± 4.2 | 11.8 ± 4.8 | ↑, P < 0.05 | |
| | [flow cytometry] | | | | | | (both sexes) | |
| | CD8+ T cells (%) | | 4.63 ± 1.2 | 3.1 ± 0.9 | 10.1 ± 0.6 | 7.8 ± 2.3 | ↑, P < 0.001 | |
| | [flow cytometry] | | | | | | (both sexes) | |
| | PCR | IFN-γ | 1.00 ± 0.25 | 0.96 ± 0.11 | 2.84 ± 0.57 | 1.31 ± 0.23 | ↑, P < 0.001 | F > M |
| | (fold) | | | | | | (female) | (P < 005) |
| | | MHC II | 1.00 ± 0.09 | 0.92 ± 0.16 | 1.57 ± 0.10 | 0.89 ± 0.30 | ↑, P < 0.001 | F > M |
| | | | | | | | (female) | (P < 0.05) |
| | | Caspase 3 | 1.00 ± 0.04 | 0.57 ± 0.07 | 1.51 ± 0.10 | 1.00 ± 0.08 | ↑, P < 0.001 | F > M |
| | | | | | | | (female) | (P < 0.001) |
| | | | | | | | ↑, P < 0.01 | |
| | | | | | | | (male) | |
| | | IL-1β | 1.00 ± 0.21 | 0.93 ± 0.03 | 0.76 ± 0.18 | 0.61 ± 0.14 | ↓, P < 0.05 | |
| | | | | | | | (both sexes) | |
| | | IL-12 | 1.00 ± 0.08 | 1.00 ± 0.07 | 2.51 ± 0.7 | 2.28 ± 0.04 | ↓, P < 0.05 | F > M |
| | | | | | | | (both sexes) | (P < 0.01) |
| | | TNF-α | 1.00 ± 0.04 | 0.29 ± 0.21 | 0.80 ± 0.10 | 0.55 ± 0.12 | ∅ | ∅ |

| | | | | | | |
|---|---|---|---|---|---|---|
| Caspase 3 activity | 2354.75 ± 871.29 | 2753.87 ± 687.26 | 7499.30 ± 692.58 | 4110.47 ± 1200.96 | ↑, P < 0.001 | F > M |
| (OD) | | | | | (female) | (P < 0.001) |
| | | | | | ↑, P < 0.01 | |
| | | | | | (male) | |

OGD = Oregon-Green-Dextran,
EGF = epidermal growth factor;
CJ = conjunctiva;
LG = lacrimal gland,
∅ = no change

Similar to the donor mice, adoptive transfer recipients of GF mice had lower frequency of Th1+ and Th17+ cells in CLN while a significant increase in Th1+ cells was observed in LG (FIG. 2C). LG lysates of GF recipients showed increased mRNA levels of MEW II, TNF-α, IL-1β, IL-23 and Caspase 3 than CON mice, indicating the cytokine milieu inside the LG became pro-inflammatory after adoptive transfer (FIG. 2D). Taken together, the results indicate that lack of commensal bacteria promotes a SS-like phenotype in GF mice that can be recapitulated in CON immunodeficient mice through greater generation of IL-12+DCs and generation of pathogenic Th1+ cells.

Microbiome Recolonization of GF Mice Reverses the Development of Lacrimal Keratoconjunctivitis

To determine if the dry eye phenotype observed in GF mice could be reversed by recolonization with commensal bacteria, the inventors used two different approaches. In the first set of experiments, 4-week old female GF mice were colonized with feces from normal mice and disease parameters were evaluated at 8 weeks of age. Adoptive transfer recipients of CD4+ T cells isolated from GF+FG mice had lower total CD4+ T cell infiltration and lower frequency of Th1+ cells in LG, demonstrating that colonization with commensals decreased generation of autoreactive CD4+ T cells (FIG. 2C).

Model 2: Desiccating Stress

In another set of animals, the inventors performed fecal gavage into mice that have been subjected to an experimental dry eye model (the desiccating stress; DS) and had previously received a cocktail of oral antibiotics (ABX). Mice drank ABX for seven days, prior to initiation of DS. On the 8th day, mice were switched normal water and DS initiated.

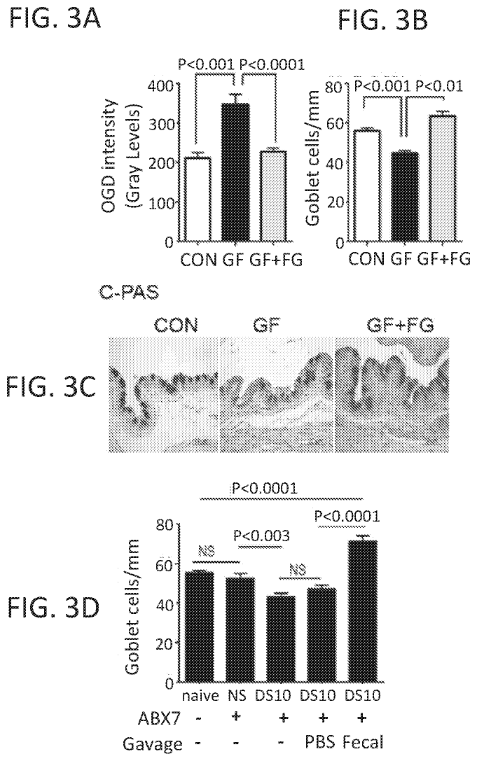

DS+ABX mice were randomized to receive oral gavage of either PBS or fecal material (fecal gavage) daily for 5 days starting at day 1 of DS. Mice were euthanized at DS10, a time point were significant goblet cell loss is observed (de Paiva et al., 2007, de Paiva et al., 2011a, Coursey et al., 2013). For this experiment, ABX treatment started 7 days prior to DS and mice drank normal water during DS because continuation of ABX during DS would affect fecal reconstitution and survival of newly transplanted bacteria. Mice that received fecal gavage during DS had a 50% increase in GCs compared to mice that received PBS gavage, demonstrating that the protective role of microbiota on conjunctival GC (FIG. 3D).

These studies clearly suggest that corneal barrier disruption and low GC density were related to lack of bacterial colonization of the gut, indicating that commensal bacteria participate on the maintenance of ocular homeostasis.

Model 3: Protective Role of Commensal Bacteria in Sjögren Syndrome Mouse Model

Microbiota is the ecological community of commensal, symbiotic and pathogenic microorganisms that literally share host body space. There are trillions of microbes in the body which account for about 1-3% of the total body mass. Microbiota help digest food, metabolism, and contribute to the maturation of the immune system and homeostasis (Ruff et al., 2015). Microbiota in the gut plays an important role in barrier against pathogens, maintenance of intestinal homeostasis and modulation of the host immune system (Hooper et al., 2012). Microbial balance and integrity are important for good health. Microbiota composition is influenced by environmental factors such as diet, antibiotic therapy and environmental exposure to microorganisms. A loss of balance (dysbiosis) can trigger digestive dysfunctions, allergies in children and chronic conditions including obesity and inflammatory diseases (Burcelin et al., 2012).

Sjögren syndrome (SS) is an autoimmune disorder that affects exocrine glands such as salivary and lacrimal glands (LG) with lymphocytic infiltration leading to dry eye and mouth. These glands have significant infiltration that results in apoptosis and acinar loss. The infiltrating cells are a mix of T-cells, B-cells, dendritic cells and natural killer cells (NK) (Christodoulou et al., 2010).

IL-2 receptor alpha chain is the binding receptor of IL-2. (Taniguchi and Minami, 1993) (CD25) knockout (KO) is a SS mouse model that recapitulates several features of SS, such as dacryoadenitis, sialodenitis, and keratoconjunctivitis (Sharma et al., 2006). These mice develop spontaneous multiorgan inflammatory disease, inclusive of exocrine glands and gastrointestinal tract, and hemolytic anemia that leads to early mortality (Willerford D M, 1995). CD25KO mice have no IL-2 signaling, have no T regulatory T cells (Tregs) and autoreactive T cells do not undergo activation cell death (Willerford D M, 1995, Sharma et al., 2005, Sharma et al., 2006). These mice develop spontaneous dacryoadenitis by 8 weeks, with 50% LG infiltration that progresses to complete atrophy by 16 weeks of age (Rahimy et al., 2010). This age-dependent LG destruction is accompanied by increased expression of T cell related cytokines. (de Paiva et al., 2010, Rahimy et al., 2010, Pelegrino et al., 2012 IFN-γ is critical in this model, as CD25-IFN-γ double-knock-out displayed delayed dacryoadenitis onset and decreased glandular apoptosis (Pelegrino et al., 2012, Bian et al., 2015).

Despite autoimmunity, CD25KO and other strains that lack Tregs are susceptible to environmental cues. It has also been shown that in young scurfy mice oral administration of LPS exacerbated salivary submandibular gland (SMG) inflammation, providing evidence that microorganisms/microbial products in the mucosa may incite the immune system and trigger autoimmunity (Sharma et al., 2006). A report showed altered eye associated lymphoid tissue in LG of GF Swiss-Webster mice, suggesting that microbiota affects mucosal LG environment (Kugadas and Gadjeva, 2016).

The purpose of this study was to investigate the role of commensal bacteria in the CD25KO murine model of SS. Herein the inventors describe that absence of microbiota in the GF model accelerates LG lymphocytic infiltration and glandular destruction. Furthermore, adoptive transfer of isolated CD4+ T cells from GF KO mice into RAG1KO mice recapitulated the dry eye phenotype observed in donor mice, demonstrating the protective role of microbiota in spontaneous dacryoadenitis in SS. Furthermore, oral gavage of fecal slurry in GF KO mice decreased generation of pathogenic Th1 cells.

Germ-Free CD25KO have Earlier Onset of Lacrimokeratoconjunctivitis than Conventional Normal Flora CD25KO

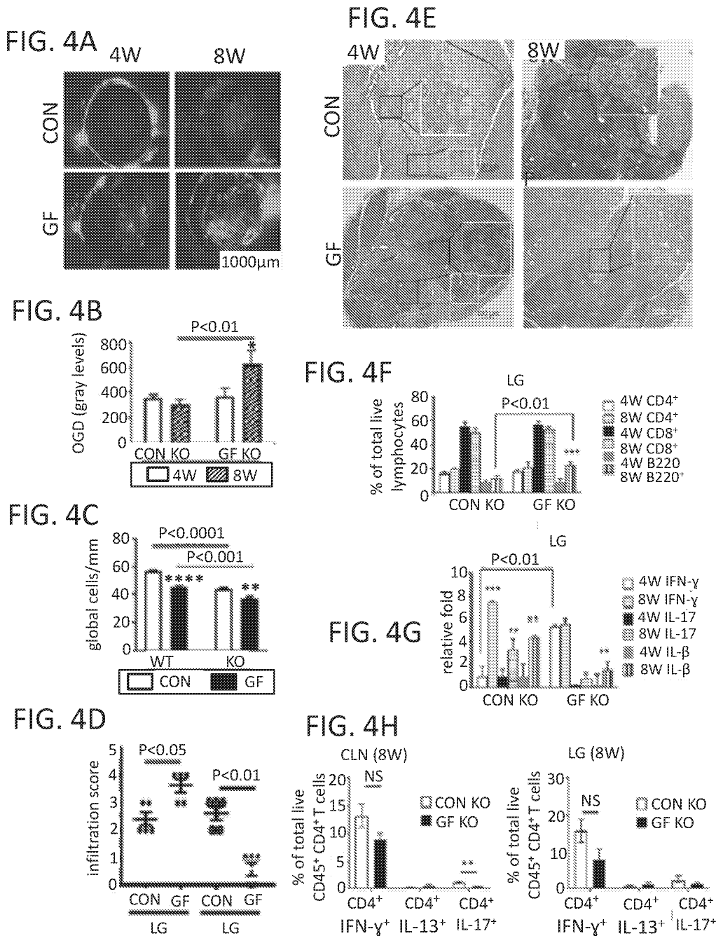

The inventors have previously shown that CD25KO mice develop lacrimokeratonconjunctivitis, with significant ocular and LG alterations (Rahimy et al., 2010, de Paiva et al., 2010, Pelegrino et al., 2012). Here the inventors investigated the role of commensal bacteria by examining the ocular and lacrimal gland phenotype in CD25KO raised in GF conditions and comparing them to CON KO mice. Because CD25KO mice have no sex predilection (Rahimy et al., 2010), these studies used mice of both sexes. It was observed that GF CD25KO mice have greater corneal barrier dysfunction and lower goblet cell density compared to CON CD25KO mice at 8 weeks of age (FIG. 4A, 4B). Total lacrimal gland infiltration was measured in histologic sections and epithelial and acinar death was graded by a masked pathologist and investigator, using a modified score described by White and Casarett (White and Casarett, 1974). Greater LG infiltration score was presented in 4-week old GF KO mice, and aging of these mice to 8 weeks further increased their total LG infiltration compared to CON KO mice (FIG. 4. (Data not shown).

LG infiltration was characterized by flow cytometry of CD4, CD8 and B220+ cells in LGs and CLN at 4 and 8 weeks of age. CD8+ T cells were the more frequent cell type irrespectively of age and housing condition (FIG. 4), followed by CD4+ T cells. There was a significant increase in frequency of B220+ cells at 8-week old GF KO in LG, but not in the draining nodes (data not shown). The phenotype of CD4+ T helper (Th) cells was investigated by intracellular staining for signature cytokines IFN-γ (Th1), IL-17 (Th17), and IL-13 (Th2). Th-1+ cells were the predominant Th subset but no differences in their frequency were observed in CLN or LG comparing both strains. On the other hand, GF KO mice had lower frequency of CD4+IL-17+ cells (Th7) in the draining LN (FIG. 4H).

The expression of inflammatory and T-cell related cytokines (IL-1β, IFN-γ, IL-17 and Caspase 3) was evaluated in LG lysates by real time PCR using the 4-week old CON KO LG as the calibrator. There was a significant early increase (4 weeks of age) in IFN-γ and caspase 3 mRNA transcripts in GF mice compared to CON (FIG. 2), paralleling the early LG infiltration seen in histologic and flow cytometry analysis.

These results suggest that commensal bacterial or products produced by them delay the onset of dacryoadenitis even in the autoimmune CD25KO mouse strain.

Greater Pathogenicity of Adoptively Transferred GF CD4+ T Cells in Immunodeficient Mice

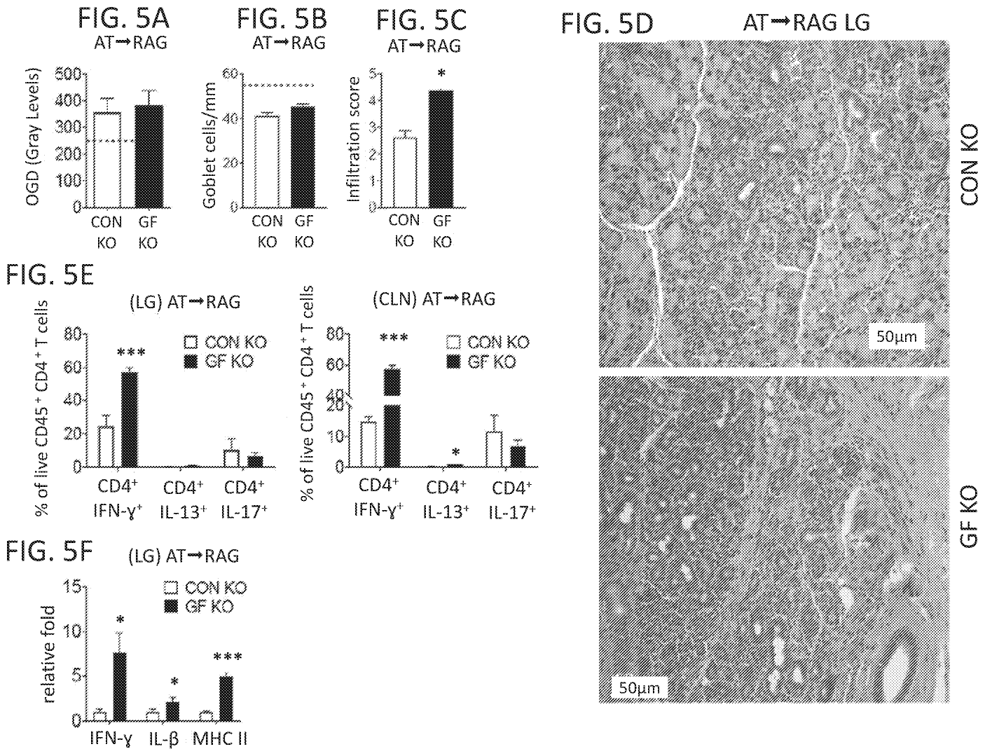

Several studies have shown that commensal bacteria play an important role in the induction of differentiation of CD4+T cells (Ruff et al., 2015). It has been previously demonstrated that adoptively transferred CD4+T cells isolated from dry eye mice produced inflammation in the lacrimal gland (Zhang et al., 2011, Niederkorn et al., 2006, de Paiva et al., 2011b). To investigate if GF KO CD4+ T cells are more pathogenic than CON KO cells, CD4+T cells were isolated from spleens and cervical lymph nodes and adoptively transferred into sex-matched RAG1KO mice. Ocular and LG inflammation in RAG1KO recipients were investigated 5 weeks post-transfer. Both GF and CON CD4+ T cell recipients showed an increased uptake of the fluorescent dye Oregon-green Dextran (OGD) used to measure corneal permeability compared to naïve RAG1KO mice, demonstrating that adoptive transfer of both cell types can equally cause barrier disruption (FIG. 5A). A similar level of goblet cell loss was seen between GF and CON KO groups, although it was lower than naïve RAG1KO levels (FIG. 5B).

However, the LG showed the greatest difference. GF KO recipients had greater LG total lymphocytic infiltration score compared with CON KO recipients (FIG. 5C, D). This was accompanied by increased cellular apoptosis and collapse of the acini; some areas of fibrosis were also present (FIG. 5D). Adoptive transfer recipients of GF KO CD4+ T cells had greater frequency of CD4+ IFN-γ+ cells in both CLN and LG while a significant increase in CD4+IL-13+ cells was noted only in CLN in this group (FIG. 5E). No difference was noted regarding the frequency of CD4+IL-17+ cells in neither CLN nor LG in any group. Increased expression of IL-13, IFN-γ, MHC II and IL-21 mRNA transcripts was observed in GF recipients compared the CON mice (FIG. 5F).

Colonization of GF KO Mice Improves Lacrimokeratoconjunctivitis and Decreases pathogenicity of CD4+ T cells.

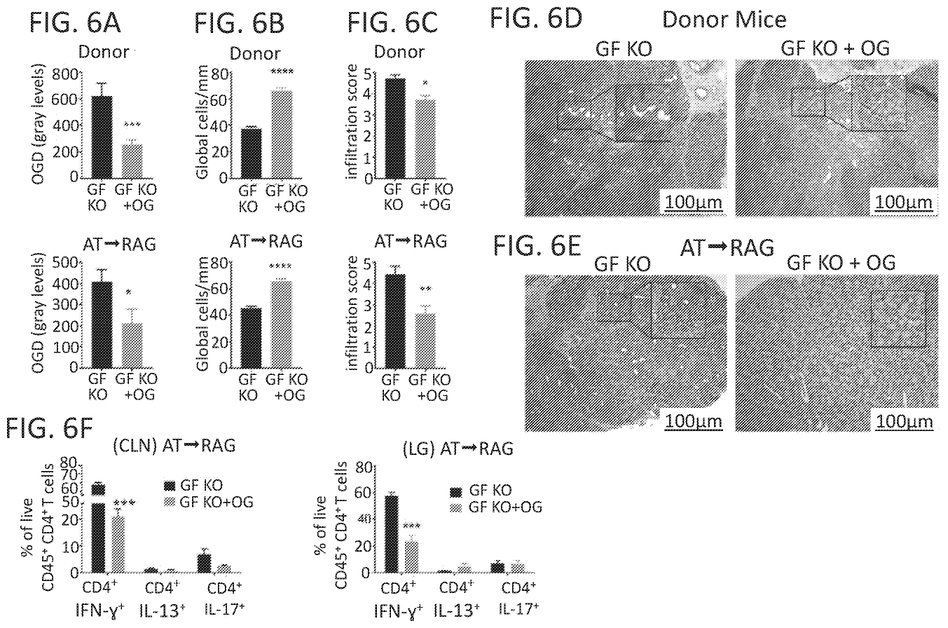

To further investigate if increased severity and early onset of lacrimokeratoconjunctivits was due to lack of commensal bacteria, the inventors performed colonization experiments by feeding GF KO mice by oral gavage (OG) a fecal slurry obtained from conventionalized C57BL/6 mice. Fecal slurries were prepared as described in the Exemplary Material and Methods and 4-week old GF KO mice were removed from the gnotobiotic incubators, received gavage, and were then housed in the general vivarium for another 4 weeks. Ocular surface and LG phenotype of GF KO+OG mice were evaluated at 8 weeks of age. GF KO mice that received oral gavage mice had a significant improvement in corneal staining, with OGD staining intensity levels similar to CON KO mice (FIG. 6A). A similar improvement was seen for goblet cell density (FIG. 6B) and lacrimal gland pathology (FIG. 6C-6D).

Because an improvement in autoimmune phenotype was observed, the inventors hypothesized that conventionalization of GF CD25KO would decrease pathogenicity of CD4+T cells. To test this hypothesis, the inventors performed adoptive transfer experiments of CD4+T cells that were isolated from GF KO that received OG and compared to recipients of GF KO CD4+ T cells. Similar to previous findings in the donor group, adoptive recipients of GF KO+OG cells had lower corneal barrier disruption, greater goblet cell density and lower LG infiltration score than GF KO recipients (FIG. 6A-6E). This was accompanied by decreased frequency of CD4+ IFN-γ+ cells in both CLN and LG of GF KO+OG recipients, demonstrating that presence of commensal bacteria in GF CD25KO mice attenuated disease phenotype and generation of pathogenic Th1 cells.

Antibiotic Treatment in Conventional CD25KO Increases Th-1 Phenotype in LG

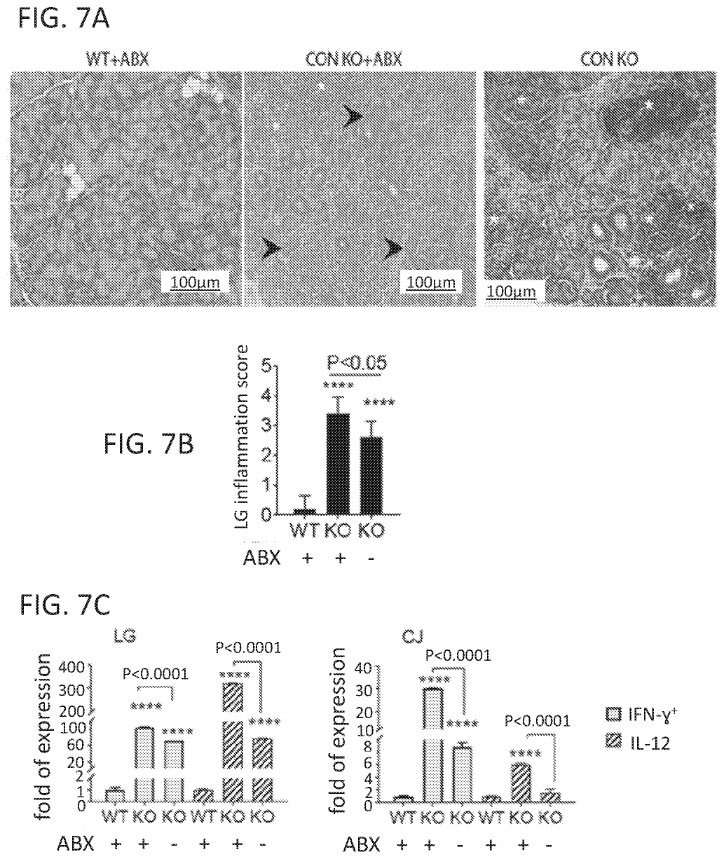

GF KO are born and raised in sterile conditions so lack of the immune system may affect the immune system development (Smith et al., 2007). Results so far have pointed out for a protective role for commensal microbiota in the CD25KO mice. To investigate if acute dysbiosis would mimic the results observed in GF KO mice, the inventors subjected CON KO mice to a cocktail of oral antibiotics (ABX) for 4 weeks starting at 4 weeks of age and compared LG pathology. Normal wild-type littermate mice that received ABX cocktail were used as naïve controls. ABX treatment in naïve WT mice had no pathogenic effect. On the other hand and in agreement with previous results, acute ablation of the microbiome in CON KO mice worsened dacryoadenitis compared to CON KO that drank normal water (FIG. 7A-7B). Gene expression in conjunctiva and LG of these mice showed a significant increase in IFN-γ and IL-12 mRNA transcripts in both tissues compared to CON CD25KO and WT+ABX mice, indicating that ABX-induced dysbiosis in CON CD25KO mice skewed autoreactive immune cells into IFN-γ producing cells (FIG. 7C).

### Example 2

### Butyrate Producing Bacteria Promote Ocular Health

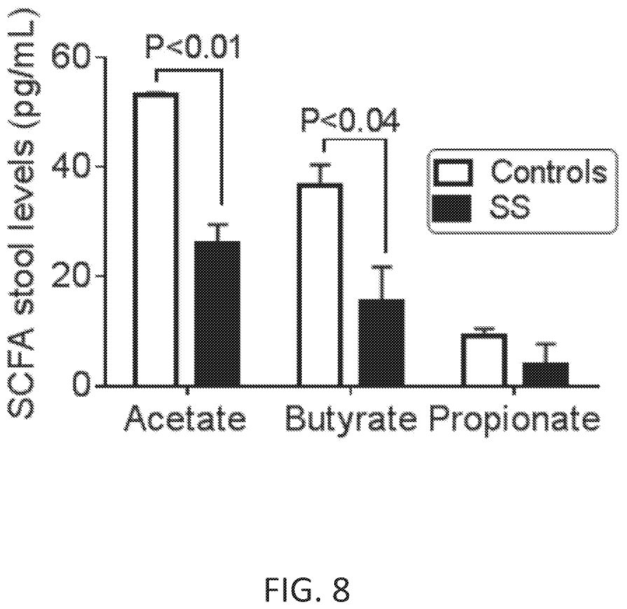

Pilot studies, now published in Scientific Reports, a journal belonging to the Nature family, compared the composition and diversity of bacteria taxa in stool samples obtained from 10 SS patients and 45 controls (de Paiva et al., 2016). 16S ribosomal RNA gene sequencing characterized the microbiota of each sample. SS subjects had greater abundances of Pseudobutyrivibrio, Escherichia, Blautia, and Streptococcus genera, but a reduced amount of Bacteroides, Parabacteroides, Faecalibacterium, and Prevotella species (de Paiva et al., 2016). There was a 50% decrease in relative abundance of OTUs classified by the NCBI database (>90% identity) to the high butyrate producer Faecalibacterium prausnitzii by NCBI mapping. Overall, the combined ocular and systemic severity score showed significant inverse correlation with microbial diversity. Of note, the conjunctival microbiome was also compared between 10 SS and 6 non-dry eye control subjects and very low abundance (the lowest of any site in the body site studied by the CMMR) with no between group differences in α or β diversity was found. (de Paiva et al., 2016) Pilot studies in FIG. 8 show lower SCFA stool levels, notably acetate and butyrate, in SS patients compared to controls, further supporting the hypothesis that alterations in bacterial communities and their metabolites impact ocular health. Despite a small sample size, the calculated power to detect differences was 99% according to the StatMate software.

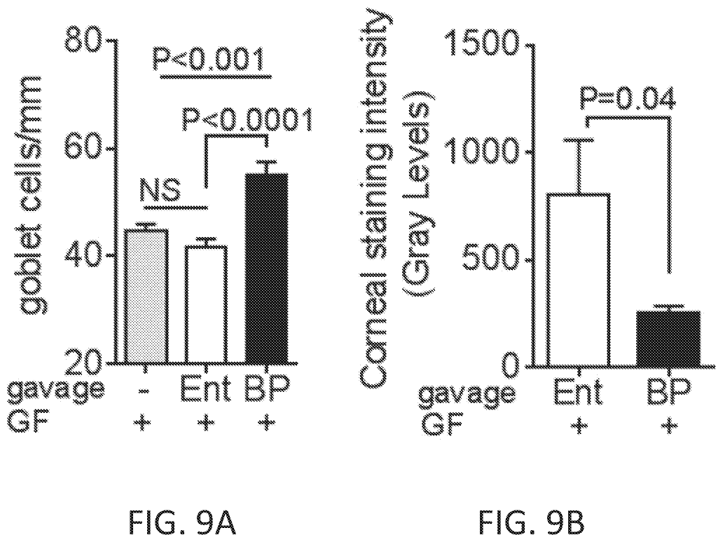

In order to test if human butyrate-producing strains promote restoration of ocular surface health, the inventors have cultivated several butyrate producing (BP) bacteria/strains from healthy human volunteers that can produce large amounts of butyrate in vitro. Dr. Britton's laboratory has a collection of over 500 microbial isolates from the human gut and is currently screening these strains for the ability to produce butyrate. The inventors have also performed a pilot study where GF mice were reconstituted with either a cocktail of three BP from his collection or with a non-BP strain (Enterococcus faecalis). Reconstitution with BP bacterial strains rescued GC and improved corneal staining 4 weeks postgavage, while reconstitution with Enterococcus faecalis did not (FIG. 9), providing evidence that BP bacteria supports ocular surface homeostasis.

### Example 3

### Exemplary Material and Methods

Animal Models.

Specific free pathogen vivarium: CD25+/− (B6.12954-IL-2ratm1Dw/J), IFN-γKO and C57BL/6 J mice breeding pairs were purchased from Jackson Laboratories (Bar Harbor, Me., USA) for establishing of breeder colonies.

Germ-Free Vivarium.

A breeder pair of CD25+/− genotype was delivery by C-section into sterile incubators at Taconic Farms and then transported into isolators to BCM germ-free facility. Heterozygous pairs of CD25 mice have been breed and housed in the vivarium in gnotobiotic incubators at Baylor College of Medicine, a GF facility, directed by Dr. Alton Sweenes.

Fecal Transplants.

Fecal slurry will be prepared by collecting fresh stools from C57BL/6 mice into a 200 ul tube containing PBS. Stool pellets will be crushed with pipette tips, and then centrifuged at 14,000 rpm for 5 mins. Supernatants will be aspired and fed into mice by oral gavage using specialized needles.

Butyrate Producing Bacteria.

Butyrate producing bacteria will be screened by their ability to produce butyrate in vitro by HPLC and they will be cultivated in standard anaerobic conditions.

Standard Desiccating Stress (DS) Model of Dry Eye.

Desiccating stress (DS) will be induced in female C57BL/6 mice aged 6-8 weeks by sterile subcutaneous injection of 0.5 mg/mL scopolamine hydrobromide (Sigma-Aldrich, St. Louis, Mo.) QID into alternating flanks and exposure to a drafty low humidity (<30% relative humidity) environment for 5 or 10 days (DS5 and DS10 respectively) as previously described (de Paiva et al., 2009). Mice subjected to this standard DS model will drink regular water.

Antibiotic Treatment and Desiccating Stress.

Six-to-eight week old female C57BL/6 mice (Jackson Labs, Bar Harbor, Me.) will be treated with a cocktail of broad-spectrum antibiotics [0.5 mg/mL Ampicillin (Dava Pharmaceuticals; Fort lee, NJ), 0.5 mg/mL Gentamicin (Life tech; Grand Islands, N.J.), 0.5 mg/mL Metronidazole (Hospira; Lake Forest, Ill.), 0.5 mg/mL Neomycin (Sparhawk lab; Lenexa, Kans.), 0.25 mg/mL Vancomycin (Hospira; Lake Forest, Ill.)] dissolved in drinking water with 5 mg/ml artificial sweetener (Splenda™, McNeil Nutritionals; Fort Washington, Pa.) as previously described (Hill et al., 2012). Mice will drink the ABX cocktail for 7 days prior to and while they will be subjected to DS for 5 or 10 days on the beginning of the 8th day.

Histology and Periodic Acid-Schiff Staining.

Right eyes and ocular adnexa were surgically excised (n=5/group), fixed in 10% formalin, paraffin embedded and will be cut into 8-μm sections. Goblet cells in sections will be stained with periodic acid-Schiff (PAS) reagent and were examined, photographed and counted with a microscope equipped with a digital camera (Eclipse E400 with a DS-Fi1; Nikon) as previously described (de Paiva et al., 2007).

Immunohistochemistry.

For immunohistochemistry, left eyes and adnexa of mice at each time point (n=5) will be excised, embedded in optimal cutting temperature (OCT compound; VWR, Suwanee, Ga.), and flash frozen in liquid nitrogen. Sagittal 8-μm sections will be cut with a cryostat (HM 500; Micron, Waldorf, Germany), placed on glass slides and stored at −80° C. The number of CD4+ T cells in the conjunctival epithelia will be counted in cryosections stained with rat-anti mouse CD4 (clone H129.9, 10 μg/mL, BD Bioscience, San Diego, Calif.) as previously described (de Paiva et al., 2007).

Measurement of Corneal Permeability.

Corneal epithelial permeability to Oregon Green Dextran (OGD; 70,000 molecular weight; Invitrogen, Eugene, Oreg.) will be assessed by instilling 0.5 μL of OGD onto the ocular surface one minute before euthanasia, as previously described (de Paiva et al., 2009). Corneas will be rinsed with PBS and photographed under fluorescence excitation at 470 nm. The severity of corneal OGD staining will be graded in digital images in the 2 mm central zone of each cornea by 2 masked observers, using the NIS Elements software (Nikon, Melville, N.Y.).

### REFERENCES

All patents and publications mentioned in the specification are indicative of the level of those skilled in the art to which the invention pertains. All patents and publications are herein incorporated by reference in their entirety to the same extent as if each individual publication was specifically and individually indicated to be incorporated by reference.
1. BIAN, F., BARBOSA, F. L., CORRALES, R. M., PELEGRINO, F. S., VOLPE, E. A., PFLUGFELDER, S. C. & DE PAIVA, C. S. 2015. Altered balance of interleukin-13/interferon-gamma contributes to lacrimal gland destruction and secretory dysfunction in CD25 knockout model of Sjogren's syndrome. Arthritis Res Ther, 17, 53.
2. BURCELIN, R., GARIDOU, L. & POMIE, C. 2012. Immuno-microbiota cross and talk: the new paradigm of metabolic diseases. Semin Immunol, 24, 67-74.
3. CHA, S., BRAYER, J., GAO, J., BROWN, V., KILLEDAR, S., YASUNARI, U. & PECK, A. B. 2004. A dual role for interferon-gamma in the pathogenesis of Sjogren's syndrome-like autoimmune exocrinopathy in the nonobese diabetic mouse. Scand. J. Immunol., 60, 552-565.
4. CHRISTODOULOU, M. I., KAPSOGEORGOU, E. K. & MOUTSOPOULOS, H. M. 2010. Characteristics of the minor salivary gland infiltrates in Sjogren's syndrome. J. Autoimmun., 34, 400-407.
5. COURSEY, T. G., GANDHI, N. B., VOLPE, E. A., PFLUGFELDER, S. C. & DE PAIVA, C. S. 2013. Chemokine receptors CCR6 and CXCR3 are necessary for CD4(+) T cell mediated ocular surface disease in experimental dry eye disease. PLoS. One., 8, e78508.
6. COURSEY, T. G., HENRIKSSON, J. T., BARBOSA, F. L., DE PAIVA, C. S. & PFLUGFELDER, S. C. 2016. Interferongamma-Induced Unfolded Protein Response in Conjunctival Goblet Cells as a Cause of Mucin Deficiency in Sjogren Syndrome. Am J Pathol, 186, 1547-58.
7. DE PAIVA, C. S., HWANG, C. S., PITCHER, J. D., III, PANGELINAN, S. B., RAHIMY, E., CHEN, W., YOON, K. C., FARLEY, W. J., NIEDERKORN, J. Y., STERN, M. E., LI, D. Q. & PFLUGFELDER, S. C. 2010. Age-related T-cell cytokine profile parallels corneal disease severity in Sjogren's syndrome-like keratoconjunctivitis sicca in CD25KO mice. Rheumatology 49, 246-258.
8. DE PAIVA, C. S., CHOITKAVANICH S, PANGELINAN S B, et al. IL-17 disrupts corneal barrier following desiccating stress. Mucosal Immunol 2009; 2:243-253
9. DE PAIVA, C. S., JONES, D. B., STERN, M. E., BIAN, F., MOORE, Q. L., CORBIERE, S., STRECKFUS, C. F., HUTCHINSON, D. S., AJAMI, N. J., PETROSINO, J. F. & PFLUGFELDER, S. C. 2016. Altered Mucosal Microbiome Diversity and Disease Severity in Sjogren Syndrome. Sci Rep, 6, 23561-23571.
10. DE PAIVA, C. S., RAINCE, J. K., MCCLELLAN, A. J., SHANMUGAM, K. P., PANGELINAN, S. B., VOLPE, E. A., CORRALES, R. M., FARLEY, W. J., CORRY, D. B., LI, D. Q. & PFLUGFELDER, S. C. 2011a. Homeostatic control of conjunctival mucosal goblet cells by NKT-derived IL-13. Mucosal. Immunol., 4, 397-408.
11. DE PAIVA, C. S., VILLARREAL, A. L., CORRALES, R. M., RAHMAN, H. T., CHANG, V. Y., FARLEY, W. J., STERN, M. E., NIEDERKORN, J. Y., LI, D. Q. & PFLUGFELDER, S. C. 2007. Dry Eye-Induced Conjunctival Epithelial Squamous Metaplasia Is Modulated by Interferon-{gamma}. Invest Ophthalmol. Vis. Sci., 48, 2553-2560.
12. DE PAIVA, C. S., VOLPE, E. A., GANDHI, N. B., ZHANG, X., ZHENG, X., PITCHER, J. D., III, FARLEY, W. J., STERN, M. E., NIEDERKORN, J. Y., LI, D. Q., FLAVELL, R. A. & PFLUGFELDER, S. C. 2011b. Disruption of TGFbeta Signaling Improves Ocular Surface Epithelial Disease in Experimental Autoimmune Keratoconjunctivitis Sicca. PLoS. One., 6, e29017. Epub 2011 Dec. 14.
13. HE, B., HOANG, T. K., WANG, T., FERRIS, M., TAYLOR, C. M., TIAN, X., LUO, M., TRAN, D. Q., ZHOU, J., TATEVIAN, N., LUO, F., MOLINA, J. G., BLACKBURN, M. R., GOMEZ, T. H., ROOS, S., RHOADS, J. M. & LIU, Y. 2017. Resetting microbiota by Lactobacillus reuteri inhibits T reg deficiency-induced autoimmunity via adenosine A2A receptors. J Exp Med, 214, 107-123.
14. HILL DA, SIRACUSA M C, ABT M C, et al. Commensal bacteria-derived signals regulate basophil hematopoiesis and allergic inflammation. Nat Med 2012; 18:538-546.
15. HOOPER, L. V., LITTMAN, D. R. & MACPHERSON, A. J. 2012. Interactions between the microbiota and the immune system. Science, 336, 1268-73.
16. ISHIMARU, N., SAEGUSA, K., YANAGI, K., HANEJI, N., SAITO, I. & HAYASHI, Y. 1999. Estrogen deficiency accelerates autoimmune exocrinopathy in murine Sjogren's syndrome through fas-mediated apoptosis. Am J Pathol, 155, 173-81.
17. KIMURA-SHIMMYO, A., KASHIWAMURA, S., UEDA, H., IKEDA, T., KANNO, S., AKIRA, S., NAKANISHI, K., MIMURA, O. & OKAMURA, H. 2002. Cytokine-induced injury of the lacrimal and salivary glands. J Immunother, 25 Suppl 1, S42-51.
18. KONG, L., ROBINSON, C. P., PECK, A. B., VELA-ROCH, N., SAKATA, K. M., DANG, H., TALAL, N. & HUMPHREYS-BEHER, M. G. 1998. Inappropriate apoptosis of salivary and lacrimal gland epithelium of immunodeficient NOD-scid mice. Clin. Exp. Rheumatol., 16, 675-681.
19. KUGADAS, A. & GADJEVA, M. 2016. Impact of Microbiome on Ocular Health. Ocul Surf.
20. MASLOWSKI, K. M., VIEIRA, A. T., NG, A., KRANICH, J., SIERRO, F., YU, D., SCHILTER, H. C., ROLPH, M. S., MACKAY, F., ARTIS, D., XAVIER, R. J., TEIXEIRA, M. M. & MACKAY, C. R. 2009. Regulation of inflammatory responses by gut microbiota and chemoattractant receptor GPR43. Nature, 461, 1282-6.
21. MCCLELLAN, A. J., VOLPE, E. A., ZHANG, X., DARLINGTON, G. J., LI, D. Q., PFLUGFELDER, S. C. & DE PAIVA, C. S. 2014. Ocular Surface Disease and Dacryoadenitis in Aging C57BL/6 Mice. Am. J. Pathol., 184, 631-43.
22. NIEDERKORN, J. Y., STERN, M. E., PFLUGFELDER, S. C., DE PAIVA, C. S., CORRALES, R. M., GAO, J. & SIEMASKO, K. 2006. Desiccating Stress Induces T Cell-Mediated Sjogren's Syndrome-Like Lacrimal Keratoconjunctivitis. J. Immunol., 176, 3950-3957.
23. PELEGRINO, F. S., VOLPE, E. A., GANDHI, N. B., LI, D. Q., PFLUGFELDER, S. C. & DE PAIVA, C. S. 2012. Deletion of interferon-gamma delays onset and severity of dacryoadenitis in CD25KO mice. Arthritis Res. Ther., 14, R234.
24. RAHIMY, E., PITCHER, J. D., III, PANGELINAN, S. B., CHEN, W., FARLEY, W. J., NIEDERKORN, J. Y., STERN, M. E., LI, D. Q., PFLUGFELDER, S. C. & DE PAIVA, C. S. 2010. Spontaneous autoimmune dacryoadenitis in aged CD25KO mice. Am J Pathol., 177, 744-753 Epub 2010 Jun. 21.
25. RUFF, W. E., VIEIRA, S. M. & KRIEGEL, M. A. 2015. The role of the gut microbiota in the pathogenesis of antiphospholipid syndrome. Curr Rheumatol Rep, 17, 472.
26. SHARMA, R., BAGAVANT, H., JARJOUR, W. N., SUNG, S. S. & JU, S. T. 2005. The role of Fas in the immune system biology of IL-2R alpha knockout mice: interplay among regulatory T cells, inflammation, hemopoiesis, and apoptosis. J. Immunol., 175, 1965-1973.
27. SHARMA, R., ZHENG, L., GUO, X., FU, S. M., JU, S. T. & JARJOUR, W. N. 2006. Novel animal models for Sjogren's syndrome: expression and transfer of salivary gland dysfunction from regulatory T celldeficient mice. J. Autoimmun., 27, 289-296.
28. SMITH, K., MCCOY, K. D. & MACPHERSON, A. J. 2007. Use of axenic animals in studying the adaptation of mammals to their commensal intestinal microbiota. Semin Immunol, 19, 59-69.
29. TANIGUCHI, T. & MINAMI, Y. 1993. The IL-2/IL-2 receptor system: a current overview. Cell, 73, 5-8.
30. TSUBOTA, K., FUKAGAWA, K., FUJIHARA, T., SHIMMURA, S., SAITO, I., SAITO, K. & TAKEUCHI, T. 1999. Regulation of human leukocyte antigen expression in human conjunctival epithelium. Invest Ophthalmol. Vis. Sci., 40, 28-34.
31. WHITE, S. C. & CASARETT, G. W. 1974. Induction of experimental autoallergic sialadenitis. J Immunol, 112, 178-85.
32. WILLERFORD DM, C. J., FERRY JA, DAVIDSON L, MA A, ALT F W. 1995. Interleukin-2 receptor alpha chain regulates the size and content of the peripheral lymphoid compartment. Immunity, 3, 521-30.
33. YOSHINO, K., MONROY, D. & PFLUGFELDER, S. C. 1996. Cholinergic stimulation of lactoferrin and epidermal growth factor secretion by the human lacrimal gland. Cornea, 15, 617-621.
34. ZHANG, X., CHEN, W., DE PAIVA, C. S., VOLPE, E. A., GANDHI, N. B., FARLEY, W. J., LI, D. Q., NIEDERKORN, J. Y., STERN, M. E. & PFLUGFELDER, S. C. 2011. Desiccating Stress Induces CD4(+) T-Cell-Mediated Sjogren's Syndrome-Like Corneal Epithelial Apoptosis via Activation of the Extrinsic Apoptotic Pathway by Interferon-gamma. Am J Pathol., 179, 1807-1814.
35. ZOUKHRI, D. 2010. Mechanisms involved in injury and repair of the murine lacrimal gland: role of programmed cell death and mesenchymal stem cells. Ocul. Surf., 8, 60-69.

1. A method of treating or preventing an autoimmune disease in an individual, comprising administering for delivery to the gastrointestinal tract of the individual a composition of microbiota, wherein said composition comprises a population of one or more microbiota capable of producing one or more short-chain fatty acids.

2. The method of claim 1, wherein the composition of one or more microbiota comprises, consists of, or consists essentially of Faecalibacterium prausnitzii, Anaerostipes, Eubacterium, Roseburia, Lactobacillus reuteri, Bacteroides, Blautia, Coprococcus, Lactobacillus casei, Lactobacillus acidophilus, Bifidobacterium bifidum, Streptococcus thermophilus, Akkermansia muciniphila, or combinations thereof.

3. The method of claim 1, wherein the composition of one or more microbiota comprises, consists of, or consists essentially of Acetanaerobacterium, Acetivibrio, Alicyclobacillus, Akkermansia, Alkaliphilus, Anaerofustis, Anaerosporobacter, Anaerostipes, Anaerotruncus, Anoxybacillus, Bacillus, Bacteroides, Blautia, Brachyspira, Brevibacillus, Bryantella, Bulleidia, Butyricicoccus, Butyrivibrio, Catenibacterium, Chlamydiales, Clostridiaceae, Clostridiales, Clostridium, Collinsella, Coprobacillus, Coprococcus, Coxiella, Deferribacteres, Desulfitobacterium, Desulfotomaculum, Dorea, Eggerthella, Erysipelothrix, Erysipelotrichaceae, Ethanoligenens, Eubacterium, Faecalibacterium, Filifactor, Flavonifractor, Flexistipes, Fulvimonas, Fusobacterium, Gemmiger, Geobacillus, Gloeobacter, Holdemania, Hydrogenoanaerobacterium, Kocuria, Lachnobacterium, Lachnospira, Lachnospiraceae, Lactobacillus, Lactonifactor, Leptospira, Lutispora, Lysinibacillus, Mollicutes, Moorella, Nocardia, Oscillibacter, Oscillospira, Paenibacillus, Papillibacter, Pseudoflavonifractor, Robinsoniella, Roseburia, Ruminococcaceae, Ruminococcus, Saccharomonospora, Sarcina, Solobacterium, Sporobacter, Sporolactobacillus, Streptomyces, Subdoligranulum, Sutterella, Syntrophococcus, Thermoanaerobacter, Thermobifida, Turicibacter, Acetonema, Amphibacillus, Ammonifex, Anaerobacter, Caldicellulosiruptor, Caloramator, Candidatus, Carboxydibrachium, Carboxydothermus, Cohnella, Dendrosporobacter Desulfitobacterium, Desulfosporosinus, Halobacteroides, Heliobacterium, Heliophilum, Heliorestis, Lachnoanaerobaculum, Oceanobacillus, Orenia (S.), Oxalophagus, Oxobacter, Pelospora, Pelotomaculum, Propionispora, Sporohalobacter, Sporomusa, Sporosarcina, Sporotomaculum, Symbiobacterium, Syntrophobotulus, Syntrophospora, Terribacillus, Thermosinus or combinations thereof.

4. The composition of claim 1, wherein the composition of microbiota may comprise, consist, or consist essentially of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 50, or no more than 100 type(s) of microbiota.

5. The composition of claim 1, wherein the composition of microbiota may comprise, consist, or consist essentially of between 1 and 100, 1 and 50, or 1 and 20; or 1 and 10, 2 and 10, 3 and 10, 4 and 10, 5 and 10, 6 and 10, 7 and 10, 8 and 10, or 9 and 10; or 1 and 9, 2 and 9, 3 and 9, 4 and 9, 5 and 9, 6 and 9, 7 and 9, or 8 and 9; or 1 and 8, 2 and 8, 3 and 8, 4 and 8, 5 and 8, 6 and 8, or 7 and 8; or 1 and 7, 2 and 7, 3 and 7, 4 and 7, 5 and 7, or 6 and 7; or 1 and 6, 2 and 6, 3 and 6, 4 and 6, or 5 and 6; or 1 and 5, 2 and 5, 3 and 5, or 4 and 5; or 1 and 4, 2 and 4, or 3 and 4; or 1 and 3, or 2 and 3; or 1 and 2; or 1 type(s) of microbiota.

6. The composition of claim 1, wherein the composition comprises, consists of, or consists essentially of one type of microbiota present in amounts at least 2, 5, 10, 25, 50, 75, 100 or more than 100 times greater than any other type of microbiota present in the composition.

7. The composition of claim 1, wherein in the composition the majority of microbiota comprises, consists of, or consists essentially of Lactobacillus reuteri, Bacteroides, Blautia, and/or Coprococcus.

8. The composition of claim 1, wherein in the composition the majority of microbiota comprises, consists of, or consists essentially of two or more of Lactobacillus reuteri, Bacteroides, Blautia, or Coprococcus.

9. The composition of claim 1, wherein in the composition the majority of microbiota comprises, consists of, or consists essentially of three or more of Lactobacillus reuteri, Bacteroides, Blautia, or Coprococcus.

10. The composition of claim 1, wherein in the composition Lactobacillus reuteri is at least 25, 30, 35, 40, 45, 50, 55, 60, 65, 70, 75, 80, 85, 90, 95, 96, 97, 98, 99, or greater than 99% of the microbiota in the composition.

11. The composition of claim 1, wherein in the composition Bacteroides is at least 25, 30, 35, 40, 45, 50, 55, 60, 65, 70, 75, 80, 85, 90, 95, 96, 97, 98, 99, or greater than 99% of the microbiota in the composition.

12. The composition of claim 1, wherein in the composition Blautia is at least 25, 30, 35, 40, 45, 50, 55, 60, 65, 70, 75, 80, 85, 90, 95, 96, 97, 98, 99, or greater than 99% of the microbiota in the composition.

13. The composition of claim 1, wherein in the composition Coprococcus is at least 25, 30, 35, 40, 45, 50, 55, 60, 65, 70, 75, 80, 85, 90, 95, 96, 97, 98, 99, or greater than 99% of the microbiota in the composition.

14. The composition of claim 1, wherein the relative presence of microbiota in the composition is expressed as a ratio of a first type of microbiota to a second type of microbiota comprising, consisting of, or consisting essentially of 1:1, 1:2, 1:3, 1:4, 1:5, 1:6, 1:7, 1:8, 1:9, 1:10, 1:15, 1:20, 1:25; 1:50; 1:75, 1:100, 1:200, 1:500, 1:1000, 1:10,000, 1:100,000 or greater than 1:100,000.

15. The composition of claim 1, wherein the concentration of a given microbiota or the concentration of the aggregate composition comprises 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ viable microbiota per gram of composition.

16. The method of claim 1, wherein the autoimmune disease is Sjögren syndrome.

17. The method of claim 1, wherein the autoimmune disease is one or more disease(s) comprising: Sjögren syndrome, Acute Disseminated Encephalomyelitis, Acute necrotizing hemorrhagic leukoencephalitis, Addison's disease, adhesive capsulitis, Agammaglobulinemia, Alopecia areata, Amyloidosis, Ankylosing spondylitis, Anti-GBM nephritis, Anti-TBM nephritis, Antiphospholipid syndrome, arthofibrosis, atrial fibrosis, autoimmune angioedema, autoimmune aplastic anemia, autoimmune dusautonomia, autoimmune hepatitis, autoimmune hyperlipidemia, autoimmune immunodeficiency, autoimmune inner ear disease, autoimmune myocarditis, autoimmune oophoritis, autoimmune pancreatitis, autoimmune retinopathy, autoimmune thrombocytopenic purpura, autoimmune thyroid disease, autoimmune urticaria, axonal and neuronal neuropathies, Balo disease, Behcet's disease, benign mucosal pemphigold, Bullous pemphigold, cardiomyopathy, Castleman disease, Celiac Disease, Chagas disease, chronic fatigue syndrome, chronic inflammatory demyelinating polyneuropathy, chronic Lyme disease, chronic recurrent multifocal osteomyelitis, Churg-Strauss syndrome, cicatricial pemphigold, cirrhosis, Cogans syndrome, cold agglutinin disease, congenital heart block, Coxsackle myocarditis, CREST disease, Crohn's disease, Cystic Fibrosis, essential mixed cryoglobulinemia, deficiency of the interleukin-1 receptor antagonist, demyelinating neuropathies, dermatitis herpetiformis, dermatomyosis, Devic's disease, discoid lupus, Dressler's syndrome, Dupuytren's contracture, endometriosis, endomyocardial fibrosis, eosinophilic esophagitis, eosinophilic facsciitis, erythema nodosum, experimental allergic encephalomyelitis, Evans syndrome, Familial Mediterranean Fever, fibromyalgia, fibrosing alveolitis, giant cell arteritis, giant cell myocarditis, glomerulonephritis, Goodpasture's syndrome, Graft-versus-host disease (GVHD), granulomatosus with polyanglitis, Graves' disease, Guillain-Bare syndrome, Hashimoto's encephalitis, Hashimoto's thyroiditis, hemolytic anemia, Henoch-Schonlein purpura, hepatitis, herpes gestationis, hypogammaglobulinemia, idiopathic thrombocytopenic purpura, IgA nephropathy, IgG4-related sclerosing disease, immunoregulatory lipoproteins, inclusion body myositis, inflammatory bowel disorders, interstitial cystitis, juvenile arthritis, juvenile myositis, Kawasaki syndrome, keloid, Lambert-Eaton syndrome, leukocytoclastic vasculitis, lichen planus, lichen sclerosus, ligneous conjunctivitis, linear IgA disease, mediastinal fibrosis, Meniere's disease, microscopic polyanglitis, mixed connective tissue disease, Mooren's ulcer, Mucha-Hamermann disease, Multiple Sclerosis (MS), Myasthenia gravis, myelofibrosis, Myositis, narcolepsy, Neonatal Onset Multisystem Inflammatory Disease, nephrogenic systemic fibrosis, neutropenia, nonalcoholic fatty liver disease, nonalcoholic steatohepatitis (NASH), ocular-cicatricial pemphigold, optic neuritis, palindromic rheumatism, Pediatric Autoimmune Neuropsychiatric Disorders Associated with Streptococcus (PANDAS), paraneoplastic cerebellar degeneration, paroxysmal nocturnal nemoglobinuria, Parry Romberg syndrome, Parsonnage-Turner syndrome, Pars planitis, Pemphigus, Peripheral neuropathy, perivenous encephalomyelitis, pernicious anemia, Peyronie's disease, POEMS syndrome, polyarteritis nodosa, progressive massive fibrosis, Tumor Necrosis Factor Receptor-associated Periodic Syndrome, Type I autoimmune polyglandular syndrome, Type II autoimmune polyglandular syndrome, Type III autoimmune polyglandular syndrome, polymyalgia rhematica, polymyositis, postmyocardial infarction syndrome, postpericardiotomy syndrome, progesterone dermatitis, primary biliary cirrhosis, primary sclerosing cholangitis, psoriasis, psoriatic arthritis, idiopathic pulmonary fibrosis, pyoderma gangrenosum, pure red cell aplasia, Raynauds phenomenon, reactic arthritis, reflex sympathetic dystrophy, Reiter's syndrome, relapsing polychondritis, restless legs syndrome, retroperitoneal fibrosis, rheumatic fever, rheumatoid arthritis, sarcoidosis, Schmidt syndrome, scleritis, scleroderma, sperm and testicular autoimmunity, stiff person syndrome, subacute bacterial endocarditis, Susac's syndrome, sympathetic ophthalmia, systemic lupus erythematosus (SLE), Takayasu's arthritis, temporal arteritis, thrombocytopenic purpura, Tolosa-Hunt syndrome, transverse myelitis, Type 1 diabetes, ulcerative colitis, undifferentiated connective tissue disease, uveitis, vasculitis, vesiculobullous dermatosis, and Vitiligo.

18. The method of claim 1, wherein one or more bacteria in the composition is capable of producing one or more short-chain fatty acid(s) selected from the group consisting of butyrate, acetate, propionate, valerate, and combinations thereof.

19. The composition of claim 18, wherein the one or more bacteria in the composition is capable of producing at least 1 mM, or at least 2 mM, or at least 3 mM, or at least 4 mM, or at least 5 mM, or at least 6 mM, or at least 7 mM, or at least 8 mM, or at least 9 mM, or at least 10 mM of short-chain fatty acid per gram of composition.

20. The method of claim 1, wherein the composition is comprised of microbiota derived from a fecal sample of a healthy human donor.

21. The method of claim 1, wherein the composition is formulated for oral or gastric administration to a mammalian subject.

22. A non-natural composition comprising a population of one or more microbiota capable of producing one or more short-chain fatty acids.

23. The composition of claim 22, wherein the composition of one or more microbiota comprises, consists of, or consists essentially of Faecalibacterium prausnitzii, Anaerostipes, Eubacterium, Roseburia, Lactobacillus reuteri, Bacteroides, Blautia, Coprococcus, or combinations thereof.

24. The composition of claim 22, wherein the composition of one or more microbiota comprises, consists of, or consists essentially of Acetanaerobacterium, Acetivibrio, Akkermansia, Alicyclobacillus, Alkaliphilus, Anaerofustis, Anaerosporobacter, Anaerostipes, Anaerotruncus, Anoxybacillus, Bacillus, Bacteroides, Blautia, Brachyspira, Brevibacillus, Bryantella, Bulleidia, Butyricicoccus, Butyrivibrio, Catenibacterium, Chlamydiales, Clostridiaceae, Clostridiales, Clostridium, Collinsella, Coprobacillus, Coprococcus, Coxiella, Deferribacteres, Desulfitobacterium, Desulfotomaculum, Dorea, Eggerthella, Erysipelothrix, Erysipelotrichaceae, Ethanoligenens, Eubacterium, Faecalibacterium, Filifactor, Flavonifractor, Flexistipes, Fulvimonas, Fusobacterium, Gemmiger, Geobacillus, Gloeobacter, Holdemania, Hydrogenoanaerobacterium, Kocuria, Lachnobacterium, Lachnospira, Lachnospiraceae, Lactobacillus, Lactonifactor, Leptospira, Lutispora, Lysinibacillus, Mollicutes, Moorella, Nocardia, Oscillibacter, Oscillospira, Paenibacillus, Papillibacter, Pseudoflavonifractor, Robinsoniella, Roseburia, Ruminococcaceae, Ruminococcus, Saccharomonospora, Sarcina, Solobacterium, Sporobacter, Sporolactobacillus, Streptomyces, Subdoligranulum, Sutterella, Syntrophococcus, Thermoanaerobacter, Thermobifida, Turicibacter, Acetonema, Amphibacillus, Ammonifex, Anaerobacter, Caldicellulosiruptor, Caloramator, Candidatus, Carboxydibrachium, Carboxydothermus, Cohnella, Dendrosporobacter Desulfitobacterium, Desulfosporosinus, Halobacteroides, Heliobacterium, Heliophilum, Heliorestis, Lachnoanaerobaculum, Oceanobacillus, Orenia (S.), Oxalophagus, Oxobacter, Pelospora, Pelotomaculum, Propionispora, Sporohalobacter, Sporomusa, Sporosarcina, Sporotomaculum, Symbiobacterium, Syntrophobotulus, Syntrophospora, Terribacillus, Thermosinus or combinations thereof.

25. The composition of claim 22, wherein the composition of microbiota may comprise, consist, or consist essentially of no more than 1, no more than 2, no more than 3, no more than 4, no more than 5, no more than 6, no more than 7, no more than 8, no more than 9, no more than 10, no more than 11, no more than 12, no more than 13, no more than 14, no more than 15, no more than 16, no more than 17, no more than 18, no more than 19, no more than 20, no more than 50, or no more than 100 type(s) of microbiota.

26. The composition of claim 22, wherein the composition of microbiota may comprise, consist, or consist essentially of between 1 and 100, 1 and 50, or 1 and 20; or 1 and 10, 2 and 10, 3 and 10, 4 and 10, 5 and 10, 6 and 10, 7 and 10, 8 and 10, or 9 and 10; or 1 and 9, 2 and 9, 3 and 9, 4 and 9, 5 and 9, 6 and 9, 7 and 9, or 8 and 9; or 1 and 8, 2 and 8, 3 and 8, 4 and 8, 5 and 8, 6 and 8, or 7 and 8; or 1 and 7, 2 and 7, 3 and 7, 4 and 7, 5 and 7, or 6 and 7; or 1 and 6, 2 and 6, 3 and 6, 4 and 6, or 5 and 6; or 1 and 5, 2 and 5, 3 and 5, or 4 and 5; or 1 and 4, 2 and 4, 3 and 4; 1 and 3, 2 and 3; 1 and 2; or 1 type(s) of microbiota.

27. The composition of claim 22, wherein the composition comprises, consists of, or consists essentially of one type of microbiota present in amounts at least 2, 5, 10, 50, 100 or more than 100 times greater than any other type of microbiota present in the composition.

28. The composition of claim 22, wherein in the composition the majority of microbiota is Lactobacillus reuteri, Bacteroides, Blautia, or Coprococcus.

29. The composition of claim 22, wherein in the composition the majority of microbiota is two or more of Lactobacillus reuteri, Bacteroides, Blautia, or Coprococcus.

30. The composition of claim 22, wherein in the composition the majority of microbiota is three or more of Lactobacillus reuteri, Bacteroides, Blautia, or Coprococcus.

31. The composition of claim 22, wherein in the composition Lactobacillus reuteri is at least 25, 30, 35, 40, 45, 50, 55, 60, 65, 70, 75, 80, 85, 90, 95, 96, 97, 98, 99, or greater than 99% of the microbiota in the composition.

32. The composition of claim 22, wherein in the composition Bacteroides is at least 25, 30, 35, 40, 45, 50, 55, 60, 65, 70, 75, 80, 85, 90, 95, 96, 97, 98, 99, or greater than 99% of the microbiota in the composition.

33. The composition of claim 22, wherein in the composition Blautia is at least 25, 30, 35, 40, 45, 50, 55, 60, 65, 70, 75, 80, 85, 90, 95, 96, 97, 98, 99, or greater than 99% of the microbiota in the composition.

34. The composition of claim 22, wherein in the composition Coprococcus is at least 25, 30, 35, 40, 45, 50, 55, 60, 65, 70, 75, 80, 85, 90, 95, 96, 97, 98, 99, or greater than 99% of the microbiota in the composition.

35. The composition of claim 22, wherein the relative presence of microbiota in the composition is expressed as a ratio of a first type of microbiota to a second type of microbiota comprising, consisting of, or essentially consisting of 1:1 or a ratio of 1:2, 1:3, 1:4, 1:5, 1:6, 1:7, 1:8, 1:9, 1:10, 1:15, 1:20, 1:25; 1:50; 1:75, 1:100, 1:200, 1:500, 1:1000, 1:10,000, 1:100,000 or greater than 1:100,000.

36. The composition of claim 22, wherein the concentration of a given microbiota or the concentration of the aggregate composition comprises 1×10³, 1×10⁴, 1×10⁵, 1×10⁶, 1×10⁷, 1×10⁸, 1×10⁹, 1×10¹⁰, 1×10¹¹, 1×10¹², 1×10¹³, 1×10¹⁴, 1×10¹⁵, or greater than 1×10¹⁵ viable microbiota per gram of composition.

37. The composition of claim 22, wherein the microbial composition comprises a bacteria that produces a short chain fatty acid selected from the group consisting of butyrate, acetate, propionate, valerate, and combinations thereof.

38. The composition of claim 22, wherein the composition is capable of producing at least 1 mM, or at least 2 mM, or at least 3 mM, or at least 4 mM, or at least 5 mM, or at least 6 mM, or at least 7 mM, or at least 8 mM, or at least 9 mM, or at least 10 mM of short-chain fatty acid per gram of composition.

39. The composition of claim 22, wherein the composition is comprised of microbiota derived from a fecal sample of a healthy human donor.

40. The composition of claim 22, wherein the composition is formulated for oral or gastric administration to a mammalian subject.
